(19) United States
(12) Patent Application Publication
(10) Pub. No.: US 2026/0364079 A1
(43) Pub. Date: Nov. 19, 2026

(54) METHODS FOR TREATING IMMUNE THROMBOCYTOPENIA IN SUBJECTS WITH COGNITIVE IMPAIRMENT BY ADMINISTERING RILZABRUTINIB

(71) Applicant: Principia Biopharma Inc., Morristown, NJ (US)

(72) Inventors: Ahmed Daak, Concord, MA (US); Umer Khan, Orlando, FL (US)

(73) Assignee: Principia Biopharma Inc., Morristown, NJ (US)

(21) Appl. No.: 19/160,970

(22) PCT Filed: Dec. 8, 2023
(86) PCT No.: PCT/US2023/083090

§ 371 Date: Aug. 29, 2025

Related U.S. Application Data
(60) Provisional application No. 63/386,710, filed on Dec. 9, 2022.
(60) Provisional application No. 63/605,721, filed on Dec. 4, 2023.

(51) Int. Cl. A61K 31/519; A61K 31/4152; A61K 31/496; A61K 38/19; A61P 7/04; A61P 25/28

(52) U.S. Cl. CPC A61K 31/519; A61K 31/4152; A61K 31/496; A61K 38/196; A61P 7/04; A61P 25/28

(57) ABSTRACT

2,2-Dimethyl-3-(2-cyano-2-methoxycarbonylvinyl)cyclopropane-1-carbonyl chloride (1.8 g.) is dissolved in 10 ml. C6H6, then 1.1 g. allethrolone, 1.5 ml. pyridine, and 10 ml. C6H6 are added, the whole kept 2 days, and evapd. in vacuo to give 2.2 g. sirupy allethrolone 2,2-dimethyl-3-(2-cyano-2-methoxycarbonylvinyl)cyclopropane-1-carboxylate, n20 1.5251. Similarly prepd. are allethrolone 2,2-dimethyl-3-(2,2-dimethoxycarbonylvinyl)cyclopropane-1-carboxylate (n20 1.5110), allethrolone 2,2-dimethyl-3-(2-acetyl-2-methoxycarbonylvinyl)cyclopropane-1-carboxylate (n20 1.5136), and allethrolone 2,2-dimethyl-3-(2,2-diacetylvinyl)cyclopropane-1-carboxylate (n15 1.5160). The products are insecticides.

Disclosed herein are methods for treating immune thrombocytopenia in subjects with cognitive impairment. BTK inhibitors and pharmaceutical compositions comprising the same are also disclosed.

Immune thrombocytopenia, commonly referred to as ITP, is a rare autoimmune disease that causes high risk for bleeding, excessive bruising, and fatigue, as well as the potential for life threatening intracranial bleeding due to destruction of platelets. ITP is characterized by immune-mediated (e.g., autoantibody-mediated) platelet destruction and impaired platelet production, resulting in thrombocytopenia, a predisposition to bleeding associated with morbidity and mortality, and an adverse impact on patient quality of life (QOL).

To date, discussion about the impact of ITP on daily life and patient productivity has been investigated from within the context of health-related QOL (Cooper et al. 2021). However, fatigue, as well as memory and concentration impairment, are also common in ITP. One hypothesis is that cognitive difficulties are an indirect consequence of fatigue, corticosteroid (CS)-associated sleep disturbance, or general illness (Berti et al. 2008; Frith et al. 2012; Newton et al. 2011). However, an alternative hypothesis is that they are a direct consequence of ITP-related disruption to normal central nervous system (CNS) function, e.g., acute neural inflammation, microbleeding, and thrombotic occlusions of small blood vessels in the brain (Berti et al. 2008; Frith et al. 2012; Newton et al. 2011). Individual reports of subjective cognitive difficulties, relative to physical symptoms, have been confirmed by surveys of patients with ITP using validated self-assessment questionnaires (Broadbent et al. 1982; Fisk et al. 1994; Johns 1991; Suarez et al. 1999). In these patients, increasing cognitive difficulties were associated with increased autonomic symptom burden, fatigue, and age (Frith et al. 2012). Compared with the general population, adults with ITP report worse health-related QOL associated with multiple physical, social, and emotional effects than patients with hypertension, arthritis, or cancer (McMillan et al. 2008). Indeed, adults with ITP report that their increased fatigue and difficulties in memory and concentration reduce their ability to perform complex daily living activities, including education and employment (Frith et al. 2012; Newton et al. 2011). Despite these initial findings, there are still many open questions regarding the prevalence, nature, and severity of cognitive impairment in patients with chronic ITP.

Current therapies for adults with ITP include initial treatment with intravenous immunoglobulin (IVIG) and corticosteroids, and subsequent treatment with splenectomy, thrombopoietin receptor agonists (TPO-RAs), rituximab, fostamatinib, and immunosuppressive therapies (e.g., mycophenolate mofetil (MMF) and cyclosporine). In general, pharmacotherapy (e.g., corticosteroids, IVIG, or anti-D immunoglobulin therapy) is used for symptomatic patients with low platelet counts for reducing platelet destruction. While most patients respond initially to corticosteroids, the rate of continued remission is low. Second line therapies for ITP include rituximab and splenectomy, which are associated with risk of sepsis and immune suppression. Additionally, thrombopoietin (TPO) mimetics (Bussel 2007) are approved for the treatment of patients with chronic ITP who have not had sufficient responses to corticosteroids, IVIG, or splenectomy.

Novel, safe, and effective oral treatments to maintain platelet counts in ITP patients would represent a significant therapeutic advantage over current standard of care. By way of non-limiting example, unmet needs in relapsed and refractory ITP include: improving remission rates and durability; avoiding rapid increase of platelet counts/thrombosis risk; steroid-free regimens; and a tolerable and safe therapy that ensures good patient QOL. Thus, there is a need for novel oral therapies for treating ITP, including relapsed and refractory ITP, that address some or all of these limitations of existing therapeutic modalities.

Bruton's agammaglobulinemia tyrosine kinase (BTK) is an essential signaling element downstream of the B-cell receptor (BCR), Fc-gamma receptor (FcγR), and Fc-epsilon receptor (FcεR). BTK is a non-receptor tyrosine kinase and a member of the TEC family of kinases. BTK is essential to B cell lineage maturation, and inhibition of BTK activity in cells produces phenotypic changes consistent with blockade of the BCR. Illustratively, BTK inhibition results in the down-regulation of various B-cell activities, including cell proliferation, differentiation, maturation, and survival, and the up-regulation of apoptosis.

Rather than acting in an “on/off switch” manner, BTK may be best viewed as an immune function “modulator” (Crofford U et al., 2016; Pal Singh S et al., 2018). Important insights into BTK function come from loss of function analyses in humans and mice. Individuals with loss of function mutations in the BTK gene develop X-linked agammaglobulinemia (XLA), characterized by a complete absence of circulating B cells and plasma cells, and very low levels of immunoglobulins of all classes (Tsukada 1993, Vetrie 1993). This indicates the potential for BTK inhibition to suppress production of autoantibodies thought to be important in the development of autoimmune diseases, such as ITP.

While BTK is not expressed in T cells, natural killer cells, or plasma cells and has no traceable direct functions in T cells or plasma cells (Sideras and Smith 1995; Mohamed et al., 2009), the enzyme regulates the activation of other hematopoietic cells, such as B cells, monocytes, basophils, mast cells, macrophages, neutrophils, and platelets. For example, BTK plays a role in the activation of neutrophils, which are key players in the inflammatory response that contributes to wound healing but may also cause tissue damage (Volmering S et al., 2016).

Accordingly, a selective BTK inhibitor has the potential to target multiple pathways involved in inflammation and autoimmunity, including, but not limited to: blocking BCR signaling; inhibiting plasma cell differentiation and antibody production; blocking IgG-mediated FcγR activation, phagocytosis, and inflammatory mediators in monocytes or macrophages; blocking IgE-mediated FcFR activation and degranulation in mast cells or basophils; and inhibiting activation, adhesion, recruitment, and oxidative burst in neutrophils. Based on these effects, a selective BTK inhibitor may block the initiation and progression of various inflammatory diseases and mitigate tissue damage resulting from these diseases. Although individuals with loss of function mutations in the BTK gene have decreased humoral immunity and are susceptible to pyogenic bacterial and enterovirus infections, requiring treatment with intravenous immunoglobulin, inhibition of BTK in individuals with an intact immune system is not predicted to produce similar susceptibility to infection.

Several orally administered BTK inhibitors (BTKi), including ibrutinib (PCI-32765) and spebrutinib (CC-292), are currently marketed or in clinical development for a range of indications (Lee A et al., 2017). For example, ibrutinib has provided further clinical validation of the BTK target and was recently approved for human use in mantle cell lymphoma, Waldenstrom's macroglobulinemia, and chronic lymphocytic leukemia by the U.S. Food and Drug Administration (FDA). Ibrutinib has also demonstrated activity in other hematological malignancies (Wang 2013; Byrd 2013, Imbruvica Package Insert, 2015). In addition, CC-292 has been reported to be well tolerated in a healthy volunteer population at doses which provide 100% occupancy of the BTK enzyme (Evans 2013). Furthermore, evobrutinib recently demonstrated efficacy for multiple sclerosis in a Phase 2 trial (Montalban X et al., 2019). Other BTKi compounds are in clinical development for various immune-mediated disorders, such as rheumatoid arthritis (NCT03823378, NCT03682705, NCT03233230), and asthma (NCT03944707) (Montalban X et al., 2019; Norman P 2016; Tam C S et al., 2018; Crawford J J et al., 2018; Min T K et al., 2019; Gillooly K M 2017; Nadeem A et al., 2019).

While covalent BTKi, such as ibrutinib and acalabrutinib, improved on the selectivity issues that plagued many first-generation kinase inhibitors, these inhibitors are typically irreversible, causing permanent modification of both on- and off-target kinases and side effects such as thrombocytopenia, anemia, platelet aggregation, and hepatotoxicity (RITUXAN Prescribing Information, 2018; Drug Record Kinase Inhibitors, 2019; Khan Y et al., 2019; Paydas S, 2019; IMBRUVICA, 2013; Rigg R A et al., 2016; Tang C P S et al., 2018). Thus, there is a need for treatment modalities for immune-mediated diseases such as ITP, based on BTKi with reduced side effects.

Compound (I) is a BTK inhibitor of the following structure:

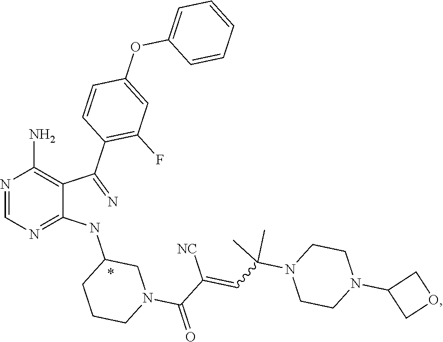

wherein *C is a stereochemical center. See PCT Publication No. WO 2014/039899, which is incorporated herein by reference, e.g., Example 31.
(R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, having the following structure:

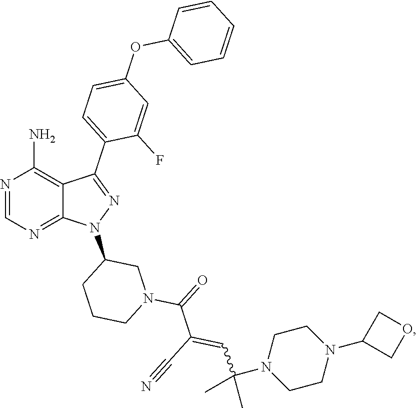 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

is also known as PRN1008 and rilzabrutinib. This compound has been disclosed in several patent publications, such as, e.g., PCT Publication Nos. WO 2014/039899, WO 2015/127310, WO 2016/100914, WO 2016/105531, WO 2018/005849, and WO 2021/150723, the contents of each of which are incorporated by reference herein.

Rilzabrutinib is a novel, highly selective, and potent small molecule inhibitor of non-T cell white blood cell signaling via B-cell receptor, FcγR, and/or FcεR signaling of the BTK pathway. In the context of ITP, rilzabrutinib has the potential to (1) inhibit B cell activation and (2) interrupt antibody-coated cell phagocytosis by FCγR in the spleen and liver (Bradshaw et al. 2021, Langrish et al. 2021, Owens et al. 2022).

Rilzabrutinib functions as a reversible covalent BTK inhibitor and is capable of both non-covalently and covalently binding to its target; in particular, its reversible cysteine binding enables high selectivity and precise BTK inhibition without a permanent modification of proteins and peptides (Langrish et al. 2021, Owens et al. 2022, Smith P F et al. 2017). Taken together, these properties allow for enhanced selectivity and extended inhibition with low systemic exposure. In comparison to first and second generation BTKi, rilzabrutinib has shown minimal cross-reactivity with other molecules and is low risk for off-target effects (Smith P F et al. 2017). Importantly, rilzabrutinib's reversible binding minimizes the likelihood of permanently modified peptides (Serafimova IM 2012). In addition, rilzabrutinib shows improved kinase selectivity relative to the covalent BTK inhibitor ibrutinib. Preclinical studies in a broad kinase enzyme inhibition panel showed that 1 μM rilzabrutinib achieved >90% inhibition of just 6 of 251 kinases sharing a common cysteine in their active site. By contrast, 1 μM ibrutinib inhibited 21 kinases. Rilzabrutinib's IC50 values were 1.3 nM for BTK, 0.8 nM for tyrosine protein kinase TEC, 1.0 nM for bone marrow tyrosine kinase on chromosome X (BMX), 1.2 nM for receptor-like kinase (RLK), 6.3 nM for B cell lymphocyte kinase (BLK), and 11 nM for ERBB4. Further preclinical assays with rilzabrutinib showed that binding to BTK persisted while that for other TEC family members decayed rapidly over time.

Rilzabrutinib has shown encouraging results for the treatment of immune-mediated diseases. In humans, rilzabrutinib is rapidly absorbed following oral administration, with a fast half-life (3-4 h) and variable pharmacokinetics (Smith P F et al., 2017).

In Phase 1 studies of rilzabrutinib with 114 healthy volunteers, target BTK occupancy levels were safely and consistently exceeded, suggesting rilzabrutinib may be highly effective in treating autoimmune diseases. Moreover, preclinical and clinical pharmacokinetic and pharmacodynamic data showed that treatment effects endured even after the compound was cleared from circulation, consistent with an extended target residence time (Hill R et al., 2015) and high target occupancy rate (>90% within four hours and high sustained occupancy over 24 hours) (Smith P F et al., 2015).

Rilzabrutinib has also demonstrated a favorable safety profile in clinical studies. In contrast with non-selective, irreversible BTK inhibitors, rilzabrutinib does not alter platelet aggregation in healthy volunteers or patients with ITP and thus does not lead to bleeding problems (Langrish et al. 2021, von Hundelshausen and Seiss 2021). As an additional point of contrast with irreversible BTK inhibitors, rilzabrutinib treatment does not exert clinically relevant effects on cardiac repolarization (electrocardiogram parameters including corrected QT interval) in healthy volunteers (N=51), even when administered at supratherapeutic doses (Lipsky and Lamanna 2020). Indeed, the most commonly reported adverse events in healthy volunteers were gastrointestinal adverse events, including nausea/vomiting and diarrhea. No serious adverse events or deaths were reported, and no participants discontinued treatment due to an adverse event (Smith P F 2017).

The efficacy of rilzabrutinib has been evaluated in both nonclinical and clinical studies. Rilzabrutinib demonstrated blockade of the rat Arthus reaction, full disease reversal in a rat collagen-induced arthritis model, and reduction in platelet loss in a mouse model of immune thrombocytopenia. The therapeutic effects of rilzabrutinib in Pemphigus was studied in dogs with newly diagnosed, naturally occurring Pemphigus foliaceus (PF). The drug safely controlled disease without the need for corticosteroid (CS) in 4 of 4 cases. The clinical efficacy of rilzabrutinib was further demonstrated in an open-label, Phase 1/2 study in immune thrombocytopenic purpura (ITP). In this trial, 40% of participants achieved the primary response endpoint after treatment with rilzabrutinib for at least 12 weeks. Furthermore, there were no related treatment-emergent serious adverse events (TESAEs) observed. Collectively, these and other studies have shown rilzabrutinib to be a safe and effective inhibitor of BTK.

There is preliminary evidence to support the role of BTK inhibition in patients with autoimmune cytopenias (Rogers 2016, Montillo 2017), where sequential episodes of severe autoimmune hemolytic anemia and ITP ceased after initiation of treatment with ibrutinib, a BTK/EGFR/ITK inhibitor, in patients with chronic lymphatic leukemia (CLL). Additionally, and pertinent to the treatment of ITP, rilzabrutinib treatment in vitro profoundly inhibits human B cell activation and blocks antibody (IgG, IgE) mediated activation of immune cells via Fc receptor signaling. In nonclinical studies, rilzabrutinib demonstrates a significant dose dependent reduction of platelet-loss (consumption) in a mouse model of ITP.

There is an unmet need for data regarding the nature, prevalence, and severity of cognitive impairment in patients with ITP, including their potential association with demographic and clinical characteristics. An ongoing phase 1/2 trial (NCT03395210) on the use of rilzabrutinib in patients with ITP therefore investigated baseline cognitive impairment in 49 patients with chronic ITP.

The study used the Cogstate Brief Battery (CBB) to investigate the following parameters: psychomotor function using the detection test (DET); visual attention using the identification test (IDN); visual learning using the one card learning test (OCL); and working memory using the one back test (ONB). Clinically important cognitive impairment was defined as a Z-score of ≤−1. When compared to age-matched normative data, both individual and composite test results provided evidence of cognitive impairment in patients with ITP. ITP patients exhibited mild impairment on tests for visual learning and working memory, as well as moderate impairment on tests of psychomotor function and attention. Overall, the severity of cognitive impairment, including the magnitude of attentional function impairment, was similar to that observed in patients with mild traumatic brain injury, moderate-to-severe depressive disorder, and chronic fatigue syndrome (Cooper N et al., 2019; Hill Q A and Newland A C, 2015; Rodeghiero F et al., 2009). The nature and severity of cognitive impairment in ITP are therefore consistent with other vascular cognitive disorders (Efficace F et al., 2016).

Disclosed herein are methods of treating immune thrombocytopenia (ITP) in a patient in need thereof comprising administering to the patient in need thereof a therapeutically effective amount of rilzabrutinib, wherein the patient in need thereof has at least one characteristic chosen from: a Z-score of ≤−1 on the Cogstate Brief Battery (CBB) detection test (DET) relative to age-matched normative data; a Z-score of ≤−1 on the CBB identification test (IDN) relative to age-matched normative data; a Z-score of ≤−1 for composite DET and IDN scores relative to age-matched normative data; a Z-score of ≤−1 on the CBB one card learning test (OCL) relative to age-matched normative data; a Z-score of ≤−1 on the CBB one back test (ONB) relative to age-matched normative data; and a Z-score of ≤−1 for composite OCL and ONB scores relative to age-matched normative data.

Also disclosed herein are methods of treating immune thrombocytopenia (ITP) in a patient in need thereof comprising administering to the patient in need thereof a therapeutically effective amount of rilzabrutinib, wherein the patient in need thereof has at least two characteristics chosen from: a Z-score of ≤−1 on the Cogstate Brief Battery (CBB) detection test (DET) relative to age-matched normative data; a Z-score of ≤−1 on the CBB identification test (IDN) relative to age-matched normative data; a Z-score of ≤−1 for composite DET and IDN scores relative to age-matched normative data; a Z-score of ≤−1 on the CBB one card learning test (OCL) relative to age-matched normative data; a Z-score of ≤−1 on the CBB one back test (ONB) relative to age-matched normative data; and a Z-score of ≤−1 for composite OCL and ONB scores relative to age-matched normative data.

### BRIEF DESCRIPTION OF DRAWINGS

FIG. 1 shows the number of patients initiating rilzabrutinib at each dose, the number of patients who completed the main study, and the number of patients who entered the long-term extension.

FIG. 2 shows a summary of intrapatient dose-escalation levels with rilzabrutinib.

FIG. 3 shows IBLS Bleeding Scale scores at baseline (Cycle 1, Day 1) and week 24/end of study for patients who completed 24 weeks of rilzabrutinib (n=34). Bleeding symptoms were grouped by site of bleeding and scored based on grade from lowest (0) to highest (3-4; grade 5 for fatal bleeding), as defined and standardized by the ITP International Working Group. There were no grade 3, 4, or 5 bleeding events at baseline or week 24/EOS; therefore, bars are not shown for these grades.

FIG. 4 shows the median platelet count over time for patients initiating rilzabrutinib at all doses, including 400 mg bid (N=60), separated by responders and nonresponders.

FIG. 5 shows the median platelet count over time for patients initiating rilzabrutinib at 400 mg bid (n=45), separated by responders and nonresponders.

FIG. 6 shows the median platelet count over time for patients initiating rilzabrutinib at all doses, including 400 mg bid (N=60).

FIG. 7 shows the median platelet count over time for patients initiating rilzabrutinib at 400 mg bid (n=45).

FIG. 8 shows rilzabrutinib dose and platelet response over time.

FIG. 9 shows the percentage of patients in each subgroup who achieved ≥2 consecutive platelet counts, separated by at least 5 days, of ≥50,000/μL, and increased ≥20,000/μL from baseline, without requiring rescue medication in the 4 weeks prior to the latest elevated platelet count.

FIG. 10 shows a summary of Z-scores for ITP patients evaluated using the Cogstate Brief Battery (CBB). Z-scores of ≤−1 (the double line) were categorized as having an impairment.

FIG. 11 shows the percentage of ITP patients exhibiting each degree of clinically important cognitive impairment on each of the individual and composite tests of the Cogstate Brief Battery (CBB).

FIG. 12 shows the percentage of ITP patients exhibiting psychomotor function/attention, learning/memory, or clinically important cognitive impairment.

FIG. 13A shows the percentage of ITP patients exhibiting psychomotor function/attention or learning/memory cognitive impairment.

FIG. 13B shows mean Z-scores for psychomotor function/attention and learning/memory in ITP patients.

FIGS. 13C and D show the mean difference between demographic and clinical characteristic factors for clinical impairment (Z-score of ≤−1) in (C) the psychomotor function/attention composite score and (D) the learning/working memory composite score. Group 1 refers to the left subgroup and Group 2 refers to the right subgroup listed in parentheses for each characteristic. Analyses were based on two-sample t-test assuming unequal variances. Note: Only 1 patient had a stroke/central nervous system bleeding, so this category was excluded. CI, confidence interval; CS, corticosteroids; ITP, immune thrombocytopenia; TPO-RA, thrombopoietin receptor agonist.

### DEFINITIONS

Unless otherwise stated, the following terms used in the specification and claims are defined for the purposes of this Application and have the following meanings. All undefined technical and scientific terms used in this Application have the meaning as commonly understood by one of ordinary skill in the art to which this disclosure belongs.

As used herein, “a” or “an” entity refers to one or more of that entity; for example, a compound refers to one or more compounds or at least one compound unless stated otherwise. As such, the terms “a” (or “an”), “one or more”, and “at least one” can be used interchangeably herein.

As used herein, the term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. With regard to specific values, it should be understood that specific values described herein for subject populations (e.g., the subject of the described clinical trial) represent median, mean, or statistical numbers, unless otherwise provided. Accordingly, aspects of the present disclosure requiring a particular value in a subject are supported herein by population data in which the relevant value is assessed to be a meaningful delimitation on the subject population.

As used herein, the term “active pharmaceutical ingredient” or “therapeutic agent” (“API”) refers to a biologically active compound.

As used herein, the term “approved treatment” refers to a medication that has received regulatory authorization, in any country, for its intended use.

As used herein, the terms “administer,” “administering,” or “administration” herein refer to providing, giving, dosing, and/or prescribing by either a health practitioner or an authorized agent and/or putting into, taking, or consuming by the patient or person himself or herself. For example, “administration” of an API to a patient refers to any route (e.g., oral delivery) of introducing or delivering the API to the patient. Administration includes self-administration and administration by another.

As used herein, the terms “baseline platelet count” or “baseline” refer to an average platelet count obtained by determining the mean of two platelet counts taken on two occasions no less than 7 days apart in the 15 days prior to beginning treatment and a third count taken on the first day of the study. If any of these three counts is missing, the “baseline platelet count” or “baseline” is the average of the other counts. As used herein, “BID” and “bid” are used interchangeably to refer to twice a day.

As used herein, the terms “cognitive impairment” or “impairment” refer to a Z-score of ≤−1 on one or more of the CBB tests. The terms “clinically important cognitive impairment” or “clinically important impairment” specifically denote at least two individual CBB tests with a Z-score of ≤−1.

As used herein, “Cogstate Brief Battery (CBB)” refers to four tests used to measure psychomotor function (the detection test (DET)), visual attention (the identification test (IDN)), visual learning (the one card learning test (OCL)), and working memory (the one back test (ONB)). The scores for these tests may be presented individually, as a composite of the psychomotor function/attention test, or as a composite of the visual learning/working memory test.

As used herein, “immune thrombocytopenia” (ITP) encompasses or at least also refers to other terms commonly used such as idiopathic thrombocytopenia and idiopathic thrombocytopenic purpura. There are three main types of ITP: acute (short term), persistent, and chronic (long term). Acute ITP lasts less than three months, persistent ITP lasts 3-12 months, and chronic ITP lasts for at least one year.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other during a treatment period. Unless specified otherwise, the two or more compounds, agents, or active pharmaceutical ingredients may be administered on different schedules during the treatment period, such as, e.g., with one or more compounds, agents, or active pharmaceutical ingredients being administered once a day and one or more other compounds, agents, or active pharmaceutical ingredients being administered twice a day.

As used herein, an amount expressed in terms of “mg of [X]” refers to the total amount in milligrams of [X], i.e., the free base. In some embodiments, rilzabrutinib may be administered as a pharmaceutically acceptable salt of rilzabrutinib, in which case an amount expressed in terms of “mg of rilzabrutinib” refers to the total amount in milligrams of rilzabrutinib, i.e., the free base, plus the equivalent amount of one or more pharmaceutically acceptable salts of rilzabrutinib based on the weight of free base therein. For example, “400 mg of at least one compound chosen from rilzabrutinib and pharmaceutically acceptable salts thereof” includes 400 mg of rilzabrutinib and a concentration of one or more pharmaceutically acceptable salts of rilzabrutinib equivalent to 400 mg of rilzabrutinib.

As used herein, a “pharmaceutically acceptable carrier or excipient” means a carrier or an excipient that is useful in preparing a pharmaceutical composition that is generally safe, and neither biologically nor otherwise undesirable, such as, e.g., a carrier or an excipient that is acceptable for mammalian pharmaceutical use.

As used herein, the term “pharmaceutically acceptable salt” refers to a salt form, e.g., an acid addition salt, of an active pharmaceutical agent that is pharmaceutically acceptable and that possesses the desired pharmacological activity of the API of which the salt is made. Pharmaceutically acceptable salts are well known in the art and include those derived from suitable inorganic and organic acids. Such salts include, but are not limited to, salts formed with inorganic acids such as hydrochloric acid, hydrobromic acid, sulfuric acid, phosphoric acid, and the like; or formed with organic acids such as formic acid, acetic acid, propionic acid, hexanoic acid, lactic acid, malonic acid, succinic acid, malic acid, maleic acid, fumaric acid, tartaric acid, citric acid, benzoic acid, mandelic acid, methanesulfonic acid, ethanesulfonic acid, 1,2-ethanedisulfonic acid, benzenesulfonic acid, 4-toluenesulfonic acid, and the like. S. M. Berge et al. describes pharmaceutically acceptable salts in detail in J. Pharmaceutical Sciences, 1977, 66, 1-19.

As used herein, the terms “PRN1008,” “rilzabrutinib,” “(R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile;” “the compound of Formula (I);” and “2-[(3R)-3-[4-amino-3-(2-fluoro-4-phenoxy-phenyl)pyrazolo[3,4-d]-pyrimidin-1-yl]piperidine-1-carbonyl]-4-methyl-4-[4-(oxetan-3-yl)piperazin-1-yl]pent-2-enenitrile” are used interchangeably to refer to a compound having the structure:

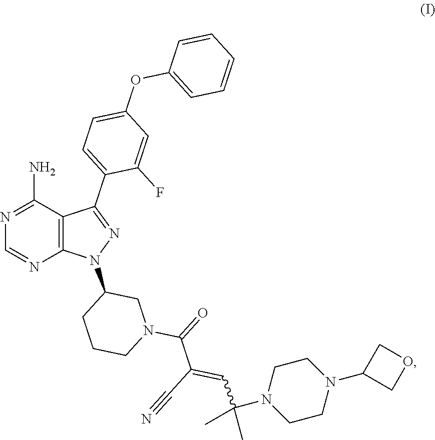 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

which is also referred to as 2-[(3R)-2-[4-amino-3-(2-fluoro-4-phenoxy-phenyl)pyrazolo[3,4-d]pyrimidin-1-yl]piperdine-1-carbonyl]-4-methyl-4[4-(oxetan-3-yl)piperazin-1-yl]-(E and Z)-pent-2-enenitrile; (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile; 1-piperidinepropanenitrile, 3-[4-amino-3-(2-fluoro-4-phenoxyphenyl)-1H-pyrazolo[3,4-d]pyrimidin-1-yl]-α-[2-methyl-2-[4-(3-oxetanyl)-1-piperazinyl]propylidene]-β-oxo-, (3R)-; (EZ)-2-[(3R)-3-[4-amino-3-(2-fluoro-4-phenoxyphenyl)pyrazolo[3,4-d]pyrimidin-1-yl]piperidine-1-carbonyl]-4-methyl-4[4-(oxetan-3-yl)piperazin-1-yl]pent-2-enenitrile; and also by the International Nonproprietary Names for Pharmaceutical Substances (INN) as published by the World Health Organization (https://cdn.who.int/media/docs/default-source/international-nonproprietary-names-(inn)//p1121.pdf?sfvrsn=69617906_15&download=true) having the following structure:

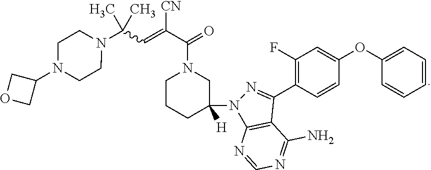 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1 ; "."]

The compound of Formula (I) includes E and Z isomers, as indicated by the wavy bond in the structure shown above. The compound of Formula (I) may be present as a salt form.

An isomer of rilzabrutinib may contain the corresponding (Z) isomer as an impurity in less than about 1% by weight; a dose of the (Z) isomer of rilzabrutinib may contain the corresponding (E) isomer as an impurity in less than about 1% by weight. When rilzabrutinib is denoted as a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, it means that the amount of (E) or (Z) isomer in the mixture is greater than about 1% by weight. In some embodiments, the molar ratio of (E) to (Z) isomer is 9:1. rilzabrutinib or a pharmaceutically acceptable salt thereof may also be referred to herein as a “drug,” “active agent,” “a therapeutically active agent,” or “API.”

As used herein, “QD” and “qd” are used interchangeably to refer to once a day.

As used herein, the term “therapeutically effective amount” refers to that an of a compound that produces the desired effect for which it is administered (e.g., improvement in ITP or a symptom of ITP, or lessening the severity of ITP or a symptom of ITP). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art.

Some embodiments of the present disclosure relate to methods of treating immune thrombocytopenia (ITP) in a patient in need thereof comprising administering to the patient in need thereof a therapeutically effective amount of rilzabrutinib, wherein the patient in need thereof has at least one characteristic chosen from: a Z-score of ≤−1 on the Cogstate Brief Battery (CBB) detection test (DET) relative to age-matched normative data; a Z-score of ≤−1 on the CBB identification test (IDN) relative to age-matched normative data; a Z-score of ≤−1 for composite DET and IDN scores relative to age-matched normative data; a Z-score of ≤−1 on the CBB one card learning test (OCL) relative to age-matched normative data; a Z-score of ≤−1 on the CBB one back test (ONB) relative to age-matched normative data; and a Z-score of ≤−1 for composite OCL and ONB scores relative to age-matched normative data.

In some embodiments, the patient has a Z-score of ≤−1 on the DET relative to age-matched normative data. In some embodiments, the patient has a Z-score of ≤−2 on the DET relative to age-matched normative data. In some embodiments, the patient has a Z-score of ≤−3 on the DET relative to age-matched normative data. In some embodiments, the patient has a Z-score of ≤−1 on the IDN relative to age-matched normative data. In some embodiments, the patient has a Z-score of ≤−2 on the IDN relative to age-matched normative data. In some embodiments, the patient has a Z-score of ≤−3 on the IDN relative to age-matched normative data. In some embodiments, the patient has a Z-score of ≤−1 for composite DET and IDN relative to age-matched normative data. In some embodiments, the patient has a Z-score of ≤−2 for composite DET and IDN relative to age-matched normative data. In some embodiments, the patient has a Z-score of ≤−3 for composite DET and IDN relative to age-matched normative data. In some embodiments, the patient has a Z-score of ≤−1 on the OCL relative to age-matched normative data. In some embodiments, the patient has a Z-score of ≤−2 on the OCL relative to age-matched normative data. In some embodiments, the patient has a Z-score of ≤−3 on the OCL relative to age-matched normative data. In some embodiments, the patient has a Z-score of ≤−1 on the ONB relative to age-matched normative data. In some embodiments, the patient has a Z-score of ≤−2 on the ONB relative to age-matched normative data. In some embodiments, the patient has a Z-score of ≤−3 on the ONB relative to age-matched normative data. In some embodiments, the patient has a Z-score of ≤−1 for composite OCL and ONB relative to age-matched normative data. In some embodiments, the patient has a Z-score of ≤−2 for composite OCL and ONB relative to age-matched normative data. In some embodiments, the patient has a Z-score of ≤−3 for composite OCL and ONB relative to age-matched normative data.

In some embodiments, the patient has a platelet count of <33,000/μL on two occasions no less than 7 days apart in the 15 days prior to beginning study treatment.

In some embodiments, the patient has a history of response (two or more platelet counts≥50,000/μL with an increase of ≥20,000/μL) to at least one prior line of therapy. In some embodiments, at least one prior line of therapy is chosen from a splenectomy, intravenous immunoglobin (IVIG), corticosteroids, anti-D immunoglobulin therapy, and immunosuppressive drugs.

In some embodiments, the patient achieves at least one platelet count of at least 20,000/μL above a baseline platelet count, wherein the baseline platelet count is the mean of two platelet counts taken on two occasions no less than 7 days apart in the 15 days prior to beginning treatment and a third count taken on the first day of the study. In some embodiments, the patient achieves at least two platelet counts of at least 20,000/μL above the baseline platelet count. In some embodiments, the patient achieves at least two consecutive platelet counts of at least 20,000/μL above the baseline platelet count, for example at least 30,000/μL above the baseline platelet count.

In some embodiments, the at least two consecutive platelet counts of at least 20,000/μL above the baseline platelet count are separated by at least five days, for example at least seven days. In some embodiments, at least two consecutive platelet counts of at least 20,000/μL above the baseline platelet count are separated by at least five days. In some embodiments, at least two consecutive platelet counts of at least 20,000/μL above the baseline platelet count are separated by at least seven days.

In some embodiments, the patient achieves a platelet count of at least 50,000/μL. In some embodiments, the patient achieves at least two platelet counts of at least 50,000/μL. In some embodiments, the patient achieves at least two consecutive platelet counts of at least 50,000/μL.

In some embodiments, the at least two consecutive platelet counts of at least 50,000/μL are separated by at least five days, for example at least seven days. In some embodiments, the at least two consecutive platelet counts of at least 50,000/μL are separated by at least five days. In some embodiments, the at least two consecutive platelet counts of at least 50,000/μL are separated by at least seven days.

In some embodiments, the patient achieves at least one platelet count of at least 50,000/μL within eight days of initiating treatment.

In some embodiments, the patient does not receive rescue medication during the four weeks prior to the most recent platelet count of at least 50,000/μL.

In some embodiments, the patient has had ITP for less than a year. In some embodiments, the patient has had ITP for one or more years. In some embodiments, the patient has had ITP for two or more years. In some embodiments, the patient has had ITP for three or more years. In some embodiments, the patient has had ITP for one or more years. In some embodiments, the patient has had ITP for four or more years. In some embodiments, the patient has had ITP for one or more years. In some embodiments, the patient has had ITP for five or more years. In some embodiments, the patient has had ITP for one or more years. In some embodiments, the patient has had ITP for six or more years. In some embodiments, the patient has had ITP for one or more years. In some embodiments, the patient has had ITP for seven or more years. In some embodiments, the patient has had ITP for one or more years. In some embodiments, the patient has had ITP for eight or more years. In some embodiments, the patient has had ITP for one or more years. In some embodiments, the patient has had ITP for nine or more years.

In some embodiments, the patient has had ITP for more than one decade. In some embodiments, the patient has had ITP for more than two decades. In some embodiments, the patient has had ITP for more than three decades. In some embodiments, the patient has had ITP for more than four decades. In some embodiments, the patient has had ITP for more than five decades.

In some embodiments, the patient has a history of taking at least one prior ITP therapy prior to the start of the treatment period. In some embodiments, the patient has a history of taking at least two prior ITP therapies prior to the start of the treatment period. In some embodiments, the patient has a history of taking at least three prior ITP therapies prior to the start of the treatment period. In some embodiments, the patient has a history of taking at least four prior ITP therapies prior to the start of the treatment period. In some embodiments, the patient has a history of taking at least five prior ITP therapies prior to the start of the treatment period. In some embodiments, the patient has a history of taking at least six prior ITP therapies prior to the start of the treatment period. In some embodiments, the patient has a history of taking at least seven prior ITP therapies prior to the start of the treatment period. In some embodiments, the patient has a history of taking at least eight prior ITP therapies prior to the start of the treatment period. In some embodiments, the patient has a history of taking at least nine prior ITP therapies prior to the start of the treatment period. In some embodiments, the patient has a history of taking at least ten prior ITP therapies prior to the start of the treatment period. In some embodiments, the patient has a history of taking at least 11 prior ITP therapies prior to the start of the treatment period. In some embodiments, the patient has a history of taking at least 12 prior ITP therapies prior to the start of the treatment period. In some embodiments, the patient has a history of taking at least 13 prior ITP therapies prior to the start of the treatment period. In some embodiments, the patient has a history of taking at least 14 prior ITP therapies prior to the start of the treatment period. In some embodiments, the patient has a history of taking at least 15 prior ITP therapies prior to the start of the treatment period. In some embodiments, the patient has a history of taking at least 16 prior ITP therapies prior to the start of the treatment period. In some embodiments, the patient has a history of taking at least 17 prior ITP therapies prior to the start of the treatment period.

In some embodiments, the patient has platelet counts between 2,000/μL and 33,000/μL on two occasions no less than 7 days apart and within 15 days prior to the start of the treatment period. In some embodiments, the patient has platelet counts≤15,000/μL on two occasions no less than 7 days apart and within 15 days prior to the start of the treatment period. In some embodiments, the patient in need thereof has platelet counts≥15,000/μL on two occasions no less than 7 days apart and within 15 days prior to the start of the treatment period.

In some embodiments, the patient has a history of response to at least one prior line of therapy, wherein at least one prior therapy is chosen from a splenectomy, intravenous immunoglobin (IVIG), corticosteroids, anti-D immunoglobulin therapy, immunosuppressive drugs, thrombopoietic receptor agonists (TPO-RAs), and rituximab. In some embodiments, the patient has a history of response to splenectomy. In some embodiments, the patient has a history of response to IVIG. In some embodiments, the patient has a history of response to corticosteroids. In some embodiments, the patient has a history of response to anti-D immunoglobulin therapy. In some embodiments, the patient has a history of response to immunosuppressive drugs. In some embodiments, the patient has a history of response to TPO-RAs. In some embodiments, the patient has a history of response to rituximab.

In some embodiments, the patient had a splenectomy prior to the start of the treatment period. In some embodiments, the patient has a history of taking intravenous immunoglobin (IVIG) prior to the start of the treatment period. In some embodiments, the patient has a history of taking corticosteroids prior to the start of the treatment period. In some embodiments, the patient has a history of taking anti-D immunoglobulin therapy prior to the start of the treatment period. In some embodiments, the patient has a history of taking at least one immunosuppressive drug prior to the start of the treatment period. In some embodiments, the patient has a history of taking at least one immunosuppressive drug is selected from fostamatinib, mycophenolate mofetil (MMF), and cyclosporine. In some embodiments, the patient has a history of taking fostamatinib prior to the start of the treatment period. In some embodiments, the patient has a history of taking MMF prior to the start of the treatment period. In some embodiments, the patient has a history of taking cyclosporine prior to the start of the treatment period. In some embodiments, the patient has a history of taking at least one TPO-RA prior to the start of the treatment period. In some embodiments, the patient has a history of taking at least one TPO-RA selected from recombinant TPO (rTPO), romiplostim, eltrombopag, and avatrombopag. In some embodiments, the patient has a history of taking rituximab prior to the start of the treatment period.

In some embodiments, the response to the prior ITP therapy comprised a platelet count of ≥50,000/μL.

In some embodiments, the patient in need thereof receives treatment with at least one TPO-RA. In some embodiments, the patient in need thereof receives treatment with at least one TPO-RA selected from rTPO, romiplostim, eltrombopag, and avatrombopag.

In some embodiments, the patient has primary ITP. In some embodiments, the patient has secondary ITP. In some embodiments, the patient does not have chronic ITP. In some embodiments, the patient has persistent ITP. In some embodiments, the patient has chronic ITP. In some embodiments, the patient has relapsing ITP. In some embodiments, the patient has refractory ITP.

In some embodiments, the treatment period is at least 8 days. In some embodiments, the treatment period is at least 28 days. In some embodiments, the treatment period is at least 84 days. In some embodiments, the treatment period is at least 169 days.

In some embodiments, the method comprises administering to the patient 400 mg of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day.

In some embodiments, the at least one compound consists of at least one compound chosen from the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof. In some embodiments, the at least one compound consists of at least one compound chosen from the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof. In some embodiments, the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt of the foregoing.

In some embodiments, the method comprises administering to the patient 400 mg of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile twice a day. In some embodiments, the method comprises administering to the patient the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile. In some embodiments, the method comprises administering to the patient the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile. In some embodiments, the method comprises administering to the patient a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.

In some embodiments, the at least one compound is orally administered to the patient. In some embodiments, the at least one compound is administered to the patient in the form of at least one tablet. In some embodiments, the at least one compound is administered with water.

Also disclosed herein are methods of treating immune thrombocytopenia (ITP) in a patient in need thereof comprising administering to the patient in need thereof a therapeutically effective amount of rilzabrutinib, wherein the patient in need thereof has at least two characteristics chosen from: a Z-score of ≤−1 on the Cogstate Brief Battery (CBB) detection test (DET) relative to age-matched normative data; a Z-score of ≤−1 on the CBB identification test (IDN) relative to age-matched normative data; a Z-score of ≤−1 for composite DET and IDN scores relative to age-matched normative data; a Z-score of ≤−1 on the CBB one card learning test (OCL) relative to age-matched normative data; a Z-score of ≤−1 on the CBB one back test (ONB) relative to age-matched normative data; and a Z-score of ≤−1 for composite OCL and ONB scores relative to age-matched normative data.

In some embodiments, the patient has at least two characteristics chosen from the following: a Z-score of <−1 on the DET test relative to age-matched normative data; a Z-score of <−1 on the IDN test relative to age-matched normative data; a Z-score of <−1 for composite DET and IDN scores relative to age-matched normative data; a Z-score of <−1 on the OCL test relative to age-matched normative data; a Z-score of <−1 on the ONB test relative to age-matched normative data; and a Z-score of <−1 for composite OCL and ONB scores relative to age-matched normative data.

In some embodiments, the patient has a Z-score of <−1 on the DET relative to age-matched normative data. In some embodiments, the patient has a Z-score of <−2 on the DET relative to age-matched normative data. In some embodiments, the patient has a Z-score of <−3 on the DET relative to age-matched normative data. In some embodiments, the patient has a Z-score of <−1 on the IDN relative to age-matched normative data. In some embodiments, the patient has a Z-score of <−2 on the IDN relative to age-matched normative data. In some embodiments, the patient has a Z-score of <−3 on the IDN relative to age-matched normative data. In some embodiments, the patient has a Z-score of <−1 for composite DET and IDN relative to age-matched normative data. In some embodiments, the patient has a Z-score of <−2 for composite DET and IDN relative to age-matched normative data. In some embodiments, the patient has a Z-score of <−3 for composite DET and IDN relative to age-matched normative data. In some embodiments, the patient has a Z-score of <−1 on the OCL relative to age-matched normative data. In some embodiments, the patient has a Z-score of <−2 on the OCL relative to age-matched normative data. In some embodiments, the patient has a Z-score of <−3 on the OCL relative to age-matched normative data. In some embodiments, the patient has a Z-score of <−1 on the ONB relative to age-matched normative data. In some embodiments, the patient has a Z-score of <−2 on the ONB relative to age-matched normative data. In some embodiments, the patient has a Z-score of <−3 on the ONB relative to age-matched normative data. In some embodiments, the patient has a Z-score of <−1 for composite OCL and ONB relative to age-matched normative data. In some embodiments, the patient has a Z-score of <−2 for composite OCL and ONB relative to age-matched normative data. In some embodiments, the patient has a Z-score of <−3 for composite OCL and ONB relative to age-matched normative data.

In some embodiments, the patient has a platelet count of <33,000/μL on two occasions no less than 7 days apart in the 15 days prior to beginning study treatment.

In some embodiments, the patient has a history of response (two or more platelet counts≥50,000/μL with an increase of ≥20,000/μL) to at least one prior line of therapy. In some embodiments, at least one prior line of therapy is chosen from a splenectomy, intravenous immunoglobin (IVIG), corticosteroids, anti-D immunoglobulin therapy, and immunosuppressive drugs.

In some embodiments, the patient achieves at least one platelet count of at least 20,000/μL above a baseline platelet count, wherein the baseline platelet count is the mean of two platelet counts taken on two occasions no less than 7 days apart in the 15 days prior to beginning treatment and a third count taken on the first day of the study. In some embodiments, the patient achieves at least two platelet counts of at least 20,000/μL above the baseline platelet count. In some embodiments, the patient achieves at least two consecutive platelet counts of at least 20,000/μL above the baseline platelet count, for example at least 30,000/μL above the baseline platelet count.

In some embodiments, the at least two consecutive platelet counts of at least 20,000/μL above the baseline platelet count are separated by at least five days, for example at least seven days. In some embodiments, at least two consecutive platelet counts of at least 20,000/μL above the baseline platelet count are separated by at least five days. In some embodiments, at least two consecutive platelet counts of at least 20,000/μL above the baseline platelet count are separated by at least seven days.

In some embodiments, the patient achieves a platelet count of at least 50,000/μL. In some embodiments, the patient achieves at least two platelet counts of at least 50,000/μL. In some embodiments, the patient achieves at least two consecutive platelet counts of at least 50,000/μL.

In some embodiments, the at least two consecutive platelet counts of at least 50,000/μL are separated by at least five days, for example at least seven days. In some embodiments, the at least two consecutive platelet counts of at least 50,000/μL are separated by at least five days. In some embodiments, the at least two consecutive platelet counts of at least 50,000/μL are separated by at least seven days.

In some embodiments, the patient achieves at least one platelet count of at least 50,000/μL within eight days of initiating treatment.

In some embodiments, the patient does not receive rescue medication during the four weeks prior to the most recent platelet count of at least 50,000/μL.

In some embodiments, the patient has had ITP for less than a year. In some embodiments, the patient has had ITP for one or more years. In some embodiments, the patient has had ITP for two or more years. In some embodiments, the patient has had ITP for three or more years. In some embodiments, the patient has had ITP for one or more years. In some embodiments, the patient has had ITP for four or more years. In some embodiments, the patient has had ITP for one or more years. In some embodiments, the patient has had ITP for five or more years. In some embodiments, the patient has had ITP for one or more years. In some embodiments, the patient has had ITP for six or more years. In some embodiments, the patient has had ITP for one or more years. In some embodiments, the patient has had ITP for seven or more years. In some embodiments, the patient has had ITP for one or more years. In some embodiments, the patient has had ITP for eight or more years. In some embodiments, the patient has had ITP for one or more years. In some embodiments, the patient has had ITP for nine or more years.

In some embodiments, the patient has had ITP for more than one decade. In some embodiments, the patient has had ITP for more than two decades. In some embodiments, the patient has had ITP for more than three decades. In some embodiments, the patient has had ITP for more than four decades. In some embodiments, the patient has had ITP for more than five decades.

In some embodiments, the patient has a history of taking at least one prior ITP therapy prior to the start of the treatment period. In some embodiments, the patient has a history of taking at least two prior ITP therapies prior to the start of the treatment period. In some embodiments, the patient has a history of taking at least three prior ITP therapies prior to the start of the treatment period. In some embodiments, the patient has a history of taking at least four prior ITP therapies prior to the start of the treatment period. In some embodiments, the patient has a history of taking at least five prior ITP therapies prior to the start of the treatment period. In some embodiments, the patient has a history of taking at least six prior ITP therapies prior to the start of the treatment period. In some embodiments, the patient has a history of taking at least seven prior ITP therapies prior to the start of the treatment period. In some embodiments, the patient has a history of taking at least eight prior ITP therapies prior to the start of the treatment period. In some embodiments, the patient has a history of taking at least nine prior ITP therapies prior to the start of the treatment period. In some embodiments, the patient has a history of taking at least ten prior ITP therapies prior to the start of the treatment period. In some embodiments, the patient has a history of taking at least 11 prior ITP therapies prior to the start of the treatment period. In some embodiments, the patient has a history of taking at least 12 prior ITP therapies prior to the start of the treatment period. In some embodiments, the patient has a history of taking at least 13 prior ITP therapies prior to the start of the treatment period. In some embodiments, the patient has a history of taking at least 14 prior ITP therapies prior to the start of the treatment period. In some embodiments, the patient has a history of taking at least 15 prior ITP therapies prior to the start of the treatment period. In some embodiments, the patient has a history of taking at least 16 prior ITP therapies prior to the start of the treatment period. In some embodiments, the patient has a history of taking at least 17 prior ITP therapies prior to the start of the treatment period.

In some embodiments, the patient has platelet counts between 2,000/μL and 33,000/μL on two occasions no less than 7 days apart and within 15 days prior to the start of the treatment period. In some embodiments, the patient has platelet counts≤15,000/μL on two occasions no less than 7 days apart and within 15 days prior to the start of the treatment period. In some embodiments, the patient in need thereof has platelet counts>15,000/μL on two occasions no less than 7 days apart and within 15 days prior to the start of the treatment period.

In some embodiments, the patient has a history of response to at least one prior line of therapy, wherein at least one prior therapy is chosen from a splenectomy, intravenous immunoglobin (IVIG), corticosteroids, anti-D immunoglobulin therapy, immunosuppressive drugs, thrombopoietic receptor agonists (TPO-RAs), and rituximab. In some embodiments, the patient has a history of response to splenectomy. In some embodiments, the patient has a history of response to IVIG. In some embodiments, the patient has a history of response to corticosteroids. In some embodiments, the patient has a history of response to anti-D immunoglobulin therapy. In some embodiments, the patient has a history of response to immunosuppressive drugs. In some embodiments, the patient has a history of response to TPO-RAs. In some embodiments, the patient has a history of response to rituximab.

In some embodiments, the patient had a splenectomy prior to the start of the treatment period. In some embodiments, the patient has a history of taking intravenous immunoglobin (IVIG) prior to the start of the treatment period. In some embodiments, the patient has a history of taking corticosteroids prior to the start of the treatment period. In some embodiments, the patient has a history of taking anti-D immunoglobulin therapy prior to the start of the treatment period. In some embodiments, the patient has a history of taking at least one immunosuppressive drug prior to the start of the treatment period. In some embodiments, the patient has a history of taking at least one immunosuppressive drug is selected from fostamatinib, mycophenolate mofetil (MMF), and cyclosporine. In some embodiments, the patient has a history of taking fostamatinib prior to the start of the treatment period. In some embodiments, the patient has a history of taking MMF prior to the start of the treatment period. In some embodiments, the patient has a history of taking cyclosporine prior to the start of the treatment period. In some embodiments, the patient has a history of taking at least one TPO-RA prior to the start of the treatment period. In some embodiments, the patient has a history of taking at least one TPO-RA selected from recombinant TPO (rTPO), romiplostim, eltrombopag, and avatrombopag. In some embodiments, the patient has a history of taking rituximab prior to the start of the treatment period.

In some embodiments, the response to the prior ITP therapy comprised a platelet count of ≥50,000/μL.

In some embodiments, the patient has primary ITP. In some embodiments, the patient has secondary ITP. In some embodiments, the patient does not have chronic ITP. In some embodiments, the patient has persistent ITP. In some embodiments, the patient has chronic ITP. In some embodiments, the patient has relapsing ITP. In some embodiments, the patient has refractory ITP.

In some embodiments, the treatment period is at least 8 days. In some embodiments, the treatment period is at least 28 days. In some embodiments, the treatment period is at least 84 days. In some embodiments, the treatment period is at least 169 days.

In some embodiments, the method comprises administering to the patient 400 mg of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day.

In some embodiments, the at least one compound consists of at least one compound chosen from the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof. In some embodiments, the at least one compound consists of at least one compound chosen from the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof. In some embodiments, the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt of the foregoing.

In some embodiments, the method comprises administering to the patient 400 mg of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile twice a day. In some embodiments, the method comprises administering to the patient the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile. In some embodiments, the method comprises administering to the patient the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile. In some embodiments, the method comprises administering to the patient a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.

In some embodiments, the at least one compound is orally administered to the patient. In some embodiments, the at least one compound is administered to the patient in the form of at least one tablet. In some embodiments, the at least one compound is administered with water.

### Pharmaceutical Compositions:

In some embodiments of the present disclosure, rilzabrutinib is administered as part of a pharmaceutical composition comprising: at least one compound chosen from rilzabrutinib and pharmaceutically acceptable salts thereof; and at least one pharmaceutically acceptable excipient. In some embodiments, the pharmaceutical composition is in the form of at least one tablet.

In some embodiments of the present disclosure, rilzabrutinib is orally administered as part of a pharmaceutical composition comprising: at least one compound chosen from rilzabrutinib and pharmaceutically acceptable salts thereof; and at least one pharmaceutically acceptable excipient. In some embodiments, the pharmaceutical composition is in the form of at least one tablet.

In some embodiments, rilzabrutinib is administered in the form of a film-coated tablet.

In some embodiments of the present disclosure, rilzabrutinib is administered in the form of at least one tablet comprising: at least one compound chosen from rilzabrutinib and pharmaceutically acceptable salts thereof; and at least one pharmaceutically acceptable excipient. In some embodiments, rilzabrutinib is administered in the form of at least one tablet comprising: at least one compound chosen from rilzabrutinib and pharmaceutically acceptable salts thereof; at least one filler; at least one disintegrant; at least one lubricant; and at least one film coating.

In some embodiments, rilzabrutinib is administered with a glass of water.

The proportion and nature of any pharmaceutically acceptable excipient may be determined by the chosen route of administration and standard pharmaceutical practice. Except insofar as any conventional pharmaceutically acceptable excipient is incompatible with rilzabrutinib, such as by producing any undesirable biological effect or otherwise interacting in a deleterious manner with any other component(s) of the pharmaceutically composition, its use is contemplated to be within the scope of this disclosure.

Some non-limiting examples of materials which may serve as pharmaceutically acceptable excipients include: (1) sugars, such as, e.g., lactose, glucose, and sucrose; (2) starches, such as, e.g., corn starch and potato starch; (3) cellulose and its derivatives, such as, e.g., sodium carboxymethyl cellulose, ethyl cellulose, and cellulose acetate; (4) powdered tragacanth; (5) malt; (6) gelatin; (7) talc; (8) excipients, such as, e.g., cocoa butter and suppository waxes; (9) oils, such as, e.g., peanut oil, cottonseed oil, safflower oil, sesame oil, olive oil, corn oil, and soybean oil; (10) glycols, such as, e.g., propylene glycol; (11) polyols, such as, e.g., glycerin, sorbitol, mannitol, and polyethylene glycol; (12) esters, such as, e.g., ethyl oleate and ethyl laurate; (13) agar; (14) buffering agents, such as, e.g., magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York also discloses additional non-limiting examples of pharmaceutically acceptable excipients, as well as known techniques for preparing and using the same.

One skilled in the art can readily select the proper form and route of administration depending upon the disorder or condition to be treated, the stage of the disorder or condition, and other relevant circumstances.

### EXAMPLES

The following example is intended to be illustrative and is not meant in any way to limit the scope of the disclosure.

| Abbreviations: | |
|---|---|
| AE | Adverse event |
| ALP | Alkaline phosphatase |
| ALT | Alanine aminotransferase |
| ANC | Absolute neutrophil count |
| ASH | American Society of Hemotology |
| AST | Aspartate aminotransferase |
| bid/BID | Twice daily (morning and evening) |
| BP | Blood pressure |
| BTK | Bruton's Tyrosine Kinase |
| CBB | Cogstate Brief Battery |
| CBC | Complete blood count |
| CI | Confidence Interval |
| CLL | Chronic lymphocytic leukemia |
| CNS | Central nervous system |
| CPK | Creatine phosphokinase |
| CRF | Case report form |
| CS | Corticosteroids |
| CTCAE | Common Terminology Criteria for AEs |
| CYP | Cytochrome P450 |
| DET | Detection test |
| DLT | Dose-limiting toxicity |
| EC | Ethics Committee |
| ECG | Electrocardiogram |
| EQ-5D VAS | Euro-QoL 5-Dimension Visual Analog Scale |
| FSH | Follicle Stimulating Hormone |
| GFR | Glomerular Filtration Rate |
| H2 | Histamine two (receptor) |
| HCV | Hepatitis C Virus |
| HDPE | High-density polyethylene |
| HIV | Human Immunodeficiency Virus |
| HR | Heart rate |
| IBLS | ITP Bleeding Scale |
| ICH | International Conference on Harmonization |
| IDN | Identification test |
| IDSM | Independent Data Safety Monitor |
| IRB | Institutional Review Board (Human Research |
| | Ethics Committee) |
| ITP | Immune Thrombocytopenia Purpura |
| ITP-BAT | Idiopathic Thrombocytopenia Purpura |
| | Bleeding Assessment Tool |
| ITT-E | Intent-to-treat exposed |
| IVIG | Intravenous immunoglobulin |
| NK | Natural killer (cell) |
| NSAID | Non-Steroidal Anti-Inflammatory Drug |
| OCL | One card learning test |
| ONB | One back test |
| PK | Pharmacokinetic |
| PO | Oral |
| PT/INR | Prothrombin Time/International Normalized |
| | Ratio |
| PTT | Partial thromboplastin time |
| qd/QD | Once a day |
| QOL | Quality of Life |
| QTcF | QT interval corrected for heart rate (Fridiricia |
| | Correction) |
| RR | Respiratory Rate |
| SAE | Serious adverse event |
| SD | Standard deviation |
| SMC | Safety Monitoring Committee |
| TEAE | Treatment-Emergent Adverse Event |
| Tmax | Time of observed maximum plasma |
| | concentration |
| TPO | Thrombopoietin |
| TPO-RA | Thrombopoietin receptor agonist |
| t½ | Elimination half-life |
| ULN | Upper limit of normal |
| VAS | Visual analog scale |

### Example 1: An Adaptive, Open-Label, Dose-Finding, Phase 1/2 Study Investigating the Safety, Pharmacokinetics, and Clinical Activity of Rilzabrutinib, an Oral BTK Inhibitor, in Patients with Relapsed/Refractory Immune Thrombocytopenia

An ongoing phase 1/2 clinical trial (NCT03395210) investigating the safety, pharmacokinetics, and clinical activity of rilzabrutinib, an oral BTK inhibitor, in patients with relapsed/refractory immune thrombocytopenia (ITP) began enrolling patients on Mar. 22, 2018. To date, rilzabrutinib has been well-tolerated in ITP patients, with no reported treatment-related bleeding or thrombotic events. Moreover, positive preliminary results have been observed in a highly treatment-resistant and refractory patient population.

The key inclusion criteria for the phase 1/2 study are: adults aged 18-80 years old with relapsed/refractory ITP; ITP primary or secondary to other diseases (e.g., systemic lupus erythematosus, chronic lymphocytic leukemia); no other available/approved treatment options; ≥2 platelet counts≤30,000/μL at study entry¹; and adequate hematologic, hepatic, and renal function. Key exclusion criteria included: pregnant or lactating women; current drug or alcohol abuse; history of solid organ transplant; and positive screening for HIV, hepatitis B, or hepatitis C. Enrolled patients have low platelet counts, having relapsed on or been refractory to prior therapies with no available and approved therapeutic options and may continue corticosteroids and/or thrombopoietin mimetics during the study. For example, stable concomitant corticosteroid (CS) or thrombopoietin receptor agonist (TPO-RA) treatment is permitted during the study. ¹ The margin of error for platelet counts can be, for example, ±3,000/μL, depending on the equipment and methods used by the central lab. Therefore, a platelet count of 33,000/μL would be considered a protocol deviation but may not automatically disqualify a patient from the study.

The sample size employed in the study is based on clinical considerations with the intention of gaining a sufficiently high confidence level in the study results using normal approximation methods.

### Participants

Eligible immune thrombocytopenia patients were aged 18-80 years (upper age 65 years in Norway/Czech Republic) and platelet counts of <30×10⁹/L on 2 occasions no less than 7 days apart within 15 days prior to study entry. Patients were required to respond to ≥1 prior immune thrombocytopenia therapy (including splenectomy), but at baseline were unable to maintain response to prior/concomitant therapy.

### Inclusion Criteria:

The following inclusion criteria were used to inform the enrollment of patients in the phase 1/2 study, including, e.g., the dose escalation study.
1. Male and female patients, aged 18 to 80 years old (Czech Republic and Norway only: aged 18 to 65 years old)
2. Immune-related ITP (both primary and secondary)
3. Refractory or relapsed patients with no available and approved therapeutic options with a platelet count of count<30,000/μL on two occasions no less than 7 days apart in the 15 days prior to beginning study treatment
4. A history of response (two or more platelet counts≥50,000/μL with an increase of ≥20,000/μL) to at least one prior line of therapy (with splenectomy being considered a line of therapy)
5. Adequate hematologic, hepatic, and renal function (absolute neutrophil count≥1.5×10⁹/L, Hgb>9 g/dL, AST/ALT≤1.5×ULN, albumin≥3 g/dL, total bilirubin≤1.5×ULN, estimated GFR≥60 (Cockcroft and Gault method) (C1D1 pre dose may be checked up to Day −3 prior to C1D1)
6. Female patients who are of reproductive potential must agree for the duration of active treatment in the study to use a highly effective means of contraception (hormonal contraception methods that inhibits ovulation, intrauterine device, intrauterine hormone-releasing system, bilateral tubal ligation, vasectomized partner, sexual abstinence). Unless surgically sterile, postmenopausal females should have menopause confirmed by FSH testing.
7. Able to provide written informed consent and agreeable to the schedule of assessment

Additionally, participants could not commence enrollment procedures until all entry criteria had been fulfilled. Where the clinical significance of an abnormal screening test result (lab or any other tests) was uncertain, the test may have been repeated.

### Exclusion Criteria:

The following exclusion criteria were used to inform the enrollment of patients in the phase 1/2 study, including, e.g., the dose escalation study.
1. Pregnant or lactating women
2. ECG findings of QTcF>450 msec (males) or >470 msec (females), poorly controlled atrial fibrillation (i.e., symptomatic patients or a ventricular rate above 100 beats/min on ECG), or other clinically significant abnormalities
3. History or current, active malignancy requiring or likely to require chemotherapeutic or surgical treatment during the trial, with the exception of non-melanoma skin cancer
4. Transfusion with blood or blood products or plasmapheresis within 2 weeks before Day 1
5. Change in corticosteroid and/or TPO agonist dose within 2 weeks prior to Day 1 (more than 10% variation from Day 1 daily doses)
6. Use of rescue medications other than corticosteroids or TPO in exclusion #5 in the two weeks before Day 1
7. Immunosuppressant drugs other than corticosteroids—these drugs should be discontinued for at least 14 days before Day 1
8. Treatment with rituximab or splenectomy within the 3 months prior to Day 1
9. Ongoing need for the use of proton pump inhibitor drugs such as omeprazole and esomeprazole (it is acceptable to change patient to H2 receptor blocking drugs prior to Day 1)
10. Concomitant use of known strong-to-moderate inducers or inhibitors of CYP3A, including CYP3A4, within 3 days or 5 half-lives (whichever is longer) of Day 1
11. Use of CYP3A-sensitive substrate drugs with a narrow therapeutic index within 3 days or 5 half-lives (whichever is longer) of study drug dosing including, but not limited to, alfentanil, astemizole, cisapride, cyclosporine, dihydroergotamine, ergotamine, fentanyl, pimozide, quinidine, sirolimus, tacrolimus, or terfenadine
12. Planned or concomitant use of any anticoagulants and platelet aggregation inhibiting drugs such as aspirin, NSAIDs, thienopyridines (within 14 days of planned dosing through end of follow-up)
13. Has received any investigational drug within the 30 days before receiving the first dose of study medication, or at least 5 times elimination half-life of the drug (whichever is longer); patient should not be using an investigational device at the time of dosing
14. Current drug or alcohol abuse
15. Refractory nausea and vomiting, malabsorption, external biliary shunt, or significant bowel resection that would preclude adequate study drug absorption
16. History of solid organ transplant
17. Positive for screening for HIV, hepatitis B (surface and core antibodies unrelated to vaccination), or hepatitis C (anti-HCV antibody confirmed with Hep C RNA)
18. History of serious infections requiring intravenous therapy within the last 3 months before Day 1
19. Clinically significant cognitive dysfunction (≥Grade 1) or medical history suggestive of increased risk for cognitive dysfunction during the study
20. Live vaccine within 28 days prior to Day 1 or plan to receive one during the study
21. Planned surgery in the time frame of the dosing period
22. Any other clinically significant disease, condition, or medical history that, in the opinion of the Investigator, would interfere with patient safety, study evaluations, and/or study procedures

Additionally, participants must have fulfilled all entry criteria to be enrolled into the study. Participants who failed to meet the entry criteria could be rescreened once at the discretion of the Investigator after informing the study Medical Monitor.

### Trial Design

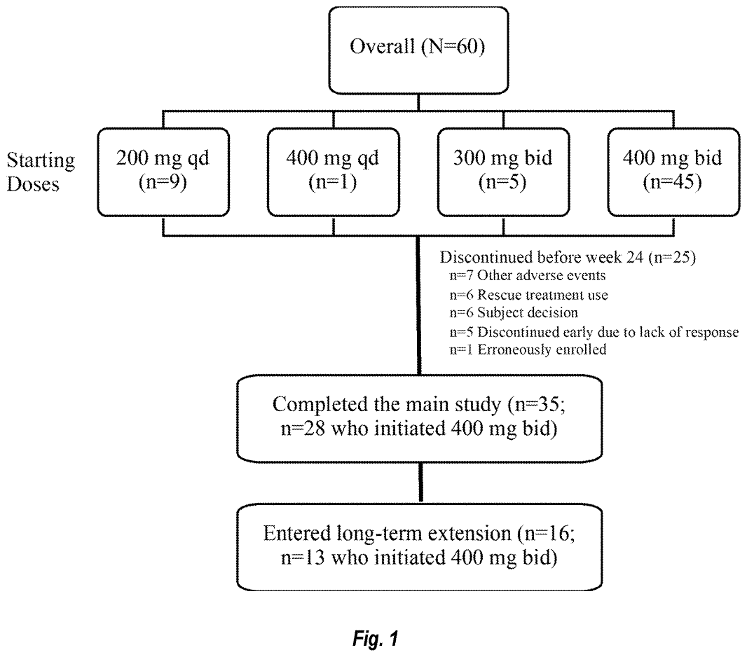

This was a global phase 1-2 adaptive, open-label, dose-finding study of oral rilzabrutinib (Principia Biopharma Inc, a Sanofi Company, South San Francisco, CA) in 8 countries (NCT03395210; EudraCT 2017-004012-19). Rilzabrutinib was administered with intrapatient dose-escalation utilizing a 3+3 design (FIG. 1). Initial doses could be 200 mg once daily (qd), 400 mg qd, 300 mg twice daily (bid; 600 mg/day), or 400 mg bid (800 mg/day; maximum). If no responses were observed at a particular dose level in 3 patients from the sentinel cohort for 28 days, the next higher dose was considered the starting dose. If response was observed at the low dose in 1 of 3 patients, then 3 more patients were added to the cohort. If a patient experienced a platelet response, then the dose was held constant at the next cycle.

Intrapatient dose-escalation was allowed every 28 days per investigator judgment to improve responses. A safety monitoring committee reviewed safety/efficacy data before all patients escalated to a higher dose. The initial study protocol specified a treatment period of 12 weeks; this was subsequently extended to 24-week treatment followed by 4-week safety follow-up. Only stable concomitant glucocorticoid/TPO-RA with ≤10% dosing change within the 2 weeks before rilzabrutinib initiation was allowed throughout treatment (unless rescue criteria were triggered). Responding patients with platelet counts≥50×10⁹/L, or ≥30×10⁹/L and doubling of baseline for ≥50% of their last 8 weeks of rilzabrutinib could continue in a long-term extension period at 400 mg bid.

Patients discontinued treatment if a dose-limiting toxicity occurred or if rescue criteria were triggered. If the patient required rescue treatment or concomitant ITP drug increases of more than 10% of the day 1 daily dose, the patient received rescue treatment per standard of care, was discontinued from the study, and was considered a nonresponder for subsequent efficacy measures.

### Assessments:

After providing informed written consent, subjects typically completed the following clinical assessments: physical examination; medical history; concomitant medications; weight; height; vital signs; ITP-BAT or IBLS bleeding scale; QOL assessment EQ-5D VAS; computerized cognitive testing using the Cogstate Brief Battery (CBB); and safety assessments.

Subjects typically completed the following laboratory and ECG assessments as part of the study:
1. Urinalysis: pH, specific gravity, protein, glucose, ketones, bilirubin, blood, nitrites, urobilinogen and leukocytes measured by dip stick or local requirement
2. Hepatitis B and C, HIV
3. Pregnancy test for women of childbearing potential only. Serum pregnancy tests at screening, urine pregnancy tests at other visits
4. FSH: To confirm postmenopausal status for women who are not surgically sterile and of reproductive potential
5. ABO and Rh blood type
6. Immature Platelet Fraction and Mean Platelet Volume (where available at local lab)
7. Serum chemistry: Aspartate aminotransferase (AST), Alanine aminotransferase (ALT), Total, direct, and indirect bilirubin levels, Alkaline phosphatase (ALP), Albumin, Creatinine, Urea, Total Protein, Sodium, Chloride, Calcium, Phosphate, Potassium, Glucose (random), and creatine phosphokinase (CPK)
8. Hematology (CBC) including differential and reticulocyte counts
9. T/B/NK/monocyte counts by flow cytometry
10. PT/INR PTT
11. TPO levels
12. Hemolysis panel consisting of Coombs test, haptoglobin levels
13. Platelet autoantibody panel (Australia Only: test excluded)
14. PK sampling at various times
15. 12-lead ECG (single and triplicate)

Laboratory assessments could be performed at both central and local laboratories, if required.

Safety assessments included the following: the frequency, severity and relationship of AEs; clinical laboratory test changes; physical examination, ECGs, vital signs, and cognitive function. AEs were coded using the Medical Dictionary for Regulatory Activities version 20.1, and severity graded according to the National Cancer Institute Common Terminology Criteria for AEs (NCI CTCAE), version 4.0. Dose-limiting toxicities (DLTs) during the rilzabrutinib treatment period were predefined in the study protocol. Hematologic DLTs included absolute neutrophil counts (ANCs) of <500/μL for ≥5 days; grade 3 or higher decreased hemoglobin in the absence of a pre-existing grade 2 decreased hemoglobin; febrile neutropenia, with absolute ANC<1000/mm³ and single temperature>38.3 °C or a sustained temperature of ≥38 °C for more than 1 h; or grade≥3 or higher bleeding event requiring platelet transfusion. Non-hematologic DLTs included any grade≥3 non-hematologic toxicity per the NCI CTCAE, version 4.0, with the following exceptions: fatigue; laboratory TEAEs, nausea, vomiting, diarrhea, or systemic reactions (such as fever, headache) that returned to baseline or grade 1 within 7 days; or any toxicity that, at the discretion of the investigator warranted withholding the study drug for >7 days.

Rescue medication (intravenous immunoglobulin (IVIG), high-dose corticosteroids, platelet infusion or anti-D immunoglobulin infusion) could be used if there was a significant deterioration in the patient's platelet count that in the opinion of the investigator put the patient at significant risk of a safety event. Patients who received rescue medication while on study were discontinued from rilzabrutinib treatment. Response status for patients who received rescue therapy while on study was evaluated based on the platelet counts up until the time of rescue therapy initiation.

In the dose escalation study, patients remained under observation in the clinic for 6 hours after administration of the first dose at the beginning of each new, higher dosing level while having intensive PK sampling performed.

### Dosage Forms:

In the study, rilzabrutinib was administered in the form a film-coated tablet. rilzabrutinib tablets are packaged in white high-density polyethylene (HDPE) bottles with child-resistant induction-sealed caps; these bottles are intended to be stored at 2-8 °C and can be transported without ice at room temperature. Additionally, the bottles can be kept at room temperature conditions for up to 2 weeks.

### Analysis Populations:

The Screening Population for this study included all participants who provided informed consent and had screening assessments evaluated for study participation.

The Safety Population included all participants who received at least one dose of rilzabrutinib. The Safety Population was used for all safety analyses. For assessment of safety by the IDSM, with regard to dropping a dose level for futility, 3 evaluable patients, defined as compliance of ≥75% of doses for that dose level, were required. During the study, patients were replaced, if necessary, to fulfill this requirement.

The Intent-to-Treat Exposed (ITT-E) Population included all participants who received at least one dose of rilzabrutinib.

The Pharmacokinetic Analysis Population included all participants who received at least one dose of rilzabrutinib and had at least one plasma concentration value that was included in the PK analysis. The Pharmacokinetic Analysis Population was used for all PK analyses.

Participants prematurely discontinued from the study, for reasons other than TEAEs, could be replaced at the discretion of the Sponsor to ensure adequate numbers of evaluable participants.

A patient who withdrew from the study before the planned end of study visit was considered to have withdrawn from the study early. Participants in this study had the right to withdraw at any time for any reason. Additionally, investigators may have withdrawn participants from the study in the event of intercurrent illness, AEs, treatment failure after a prescribed procedure, lack of compliance with the study and/or study procedures, or any other reasons where they felt it was in the best interest of the participant to be terminated from the study.

### Safety and Toxicity Management:

An Independent Data Safety Monitor (IDSM) chosen from expert clinicians in the ITP field provided independent monitoring of the phase 1/2 study. A Safety Monitoring Committee (SMC), comprised of the IDSM as Chairperson, lead Investigator, Study Medical Monitor, and Sponsor's Medical Monitor, also closely supervised the conduct of the study, meeting approximately quarterly and recommending study modification or termination to the Sponsor, based on review of safety and efficacy information. SMC findings that impacted the safety of patients in this study were reported to the local Competent Authority (CA) and IRB/EC.

The IDSM made “sentinel cohort” safety evaluations. The “sentinel patients” for each dose level had their data reviewed by the IDSM, in order to choose the starting dose for additional, new patients. After review, the IDSM could determine that a starting dose for new patients should be dropped for futility (lack of platelet response), increased to the next planned dosing level, kept the same, or reduced. New patients entering the study commenced at the dose level determined by the IDSM based on: (1) if ≥2/3 or ≥2/6 of those sentinel patients have a DLT at any dose level, that level was determined to be the “Maximally Administered Dose” and starting doses (new patients) and continuing doses (patients already on study) were set at lower dosing levels (or study suspended if the current sentinel dose cohort was 200 mg QD); (2) if two or more sustained platelet responses (3 of 4 counts) in the sentinel patients were seen at the current starting dose level the starting dose would not be escalated.

### Clinical Adverse Events

The AE Collection Period began at the time of the first screening/eligibility assessment and will end at the end of the study for each patient. An AE was defined as any untoward medical occurrence in a participant or clinical investigation participant administered a pharmaceutical product and which did not necessarily have to have a causal relationship with the intervention. An AE could therefore be any unfavorable and unintended sign (including an abnormal laboratory finding, for example), symptom, or disease temporally associated with the use of an investigational product, whether or not considered related to the product. Investigators were instructed to record all AEs encountered during the clinical study in detail from the date of participant consent throughout the study follow-up period. Pre-existing conditions that worsened during a study were reported as AEs.

### Adverse Event Relationship to Study Drug

Investigators were instructed to use their knowledge of the study participant, the circumstances surrounding the event, and an evaluation of any potential alternative causes to determine whether an AE could have been considered as related to the study drug, indicating “yes” or “no” accordingly. Investigators were asked to consider following information in assessing relatedness: (1) temporal relationship of event onset to the initiation of study drug; (2) course of the event, considering especially the effects of dose reduction, discontinuation of study drug, or reintroduction of study drug (if applicable); (3) known association of the event with the study drug or with similar treatments; (4) known association of the event with the disease under study; (5) presence of risk factors in the study participant or use of concomitant medications known to increase the occurrence of the events; and (6) presence of non-treatment-related factors that were known to be associated with the occurrence of the event.

Investigators were instructed to follow up AEs, especially those for which the severity is Grade 3 or higher, until stabilization or until 4 weeks post last dose (considered as the last follow up), based on the PK profile of the drug.

### Laboratory and ECG Abnormalities

Investigators were instructed to record any treatment-emergent abnormal laboratory or ECG result that is clinically significant, i.e., meeting one or more of the following conditions, as a single diagnosis on the AE page in the CRF. As non-limiting examples, laboratory and ECG abnormalities accompanied by clinical symptoms, leading to a change in study drug (e.g., dose modification, interruption or permanent discontinuation), or requiring a change in concomitant therapy (e.g., addition of, interruption of, discontinuation of, or any other change in a concomitant medication, therapy or treatment) should have been recorded as an AE, with any laboratory or ECG result abnormality fulfilling the criteria for a serious adverse event (SAE) reported as such, in addition to being recorded as an AE.

### Adverse Event Intensity Grading

Investigators were instructed to report all clinical AEs encountered during the study. The intensity of AEs is graded based on the NCI CTCAE, Version 4.0 or higher. For any AEs not found in the CTCAE, a description of intensity grading can be found below:
Grade 1: Mild; asymptomatic or mild symptoms; clinical or diagnostic observations only; intervention not indicated.
Grade 2: Moderate; minimal, local or noninvasive intervention indicated; limiting age-appropriate instrumental activities of daily living.
Grade 3: Severe or medically significant but not immediately life-threatening; hospitalization or prolongation of hospitalization indicated; disabling; limiting self-care activities of daily living.

A serious adverse event (SAE) was defined as any experience (clinical AE or abnormal laboratory test) that suggested a significant hazard, contraindication, side effect, or precaution. An SAE must have fulfilled at least one of the following criteria at any dose level: was fatal (results in the outcome death); was life-threatening; required in-patient hospitalization or prolongation of existing hospitalization; resulted in persistent or significant disability/incapacity; was a congenital anomaly/birth defect; or was medically significant or requires intervention to prevent one or other of the outcomes listed above. Investigators were instructed to report life-threatening events or any event with an outcome of death should be reported as an SAE.

### Pregnancy

Any female clinical trial participant who became pregnant during the study was to be instructed to stop taking the study drug and immediately inform the Investigator. Pregnancies occurring up to 90 days after the completion of the study drug were also to be reported to the Investigator.

### Ethical Considerations:

Investigators were tasked with ensuring that the study was conducted in full conformance with the principles of the “Declaration of Helsinki” or with the laws and regulations of the country in which the research is conducted, whichever affords the greater protection to the individual. Additionally, the study adheres to the principles outlined in current “Guideline for Good Clinical Practice” ICH Tripartite Guideline or with local law if it affords greater protection to the participant.

Signed and dated informed consent was obtained from each participant prior to participating in the study after adequate explanation of the aims, methods, objectives and potential hazards of the study. The Investigator or designee must have explained that the participants were completely free to refuse to enter the study or to withdraw from it at any time, for any reason.

### Physical Examination Procedures:

At screening and follow-up visits in the study, a complete physical examination consisted of checking the normality or abnormality of the following body systems: general appearance, skin, eyes, ears, nose, throat, heart, chest/breast, abdomen, neurological system, lymph nodes, spine and extremities (skeletal) and the conduct of computerized cognitive testing of learning and memory (the Cogstate Brief Battery (CBB)). An abbreviated physical examination consisted of checking the normality or abnormality of the following body systems: general appearance, skin, abdomen, and cardiorespiratory examination. Height was recorded at screening only. Blood pressure (BP), pulse rate, body temperature and respiratory rate were recorded at specific time points.

Single 12-lead ECG assessments were also obtained at specific time points to confirm eligibility and to ensure real time safety evaluation of the participants in the study. For ECG evaluations, participants should have been in a resting position for at least 10 minutes prior to any measurement. Body position should also have been consistently maintained for each ECG evaluation. In particular, changes in heart rate should have been avoided. There should have been no environmental distractions (TV, radio, conversation) during the pre-ECG rest and the ECG recording time.

Heart rate (HR), QRS duration and respiratory rate (RR), and QT intervals were recorded. Changes of the T-wave and U-wave morphology and overall ECG interpretation were documented. All ECG recordings were performed using a standard high-quality, high-fidelity digital electrocardiograph machine equipped with computer-based interval measurements. For triplicate ECG assessments, at least three interpretable ECG recordings (without artifacts) were collected per time point within a ±10 minute period per time point.

### Laboratory Test Procedures:

Laboratory assessments were performed at a central laboratory, with the provision for occasional local laboratory testing, if required. Laboratory safety tests were collected at specific time points. Additional blood or urine samples may have been taken at an investigator's discretion if the results of any test fell outside the reference ranges, or clinical symptoms necessitated additional testing to monitor participant safety. Where the clinical significance of abnormal lab results was considered uncertain, screening lab tests may have been repeated before Day 1 to confirm eligibility. In the event of unexplained abnormal clinically significant laboratory test values, the tests should have been repeated immediately and followed up until they returned to the normal range, were considered to be clinically stable, and/or an adequate explanation of the abnormality was found.

### Outcomes:

The primary study endpoints were safety and platelet count response. Safety was graded per NCI CTCAE, version 4.0. Platelet response was defined as the proportion of patients achieving ≥2 consecutive platelet counts (separated by ≥5 days) of ≥50×10⁹/L and increased ≥20×10⁹/L from baseline without immune thrombocytopenia rescue medication use in the 4 weeks before the latest elevated platelet count. In cases where a platelet count was missing, the average of non-missing platelet counts was used.

The secondary efficacy endpoints were the percent of weeks with platelet counts≥50×10⁹/L, the proportion of patients with 4 out of the final 8 platelet counts≥50×10⁹/L, change from baseline to the average of the post day 1 platelet counts for patients with ≥4 weeks of treatment, number of weeks with platelet counts≥50×10⁹/L, number of weeks with platelet counts≥30×10⁹/L, and time to first platelet count≥50×10⁹/L. Secondary safety endpoints included rescue medication use, proportion of patients with grade≥2 bleeding events, and bleeding scale scores (using the immune thrombocytopenia-specific bleeding assessment tool (ITP-BAT) (Rodeghiero et al., 2013) and/or immune thrombocytopenia bleeding scale (IBLS)) at the end of the treatment period. Post-hoc analyses are included for responders and nonresponders, as well as subgroup analyses of platelet response that evaluated the potential impact of baseline patient and disease characteristics.

### Statistical Analyses:

The safety population included all patients receiving ≥1 dose of rilzabrutinib. The intent-to-treat population included all patients who enrolled into the study. With an initial sample size of 24 and expected efficacy rate of 40% (i.e., platelet response in 15 evaluable patients), the study could yield 80% confidence that the true proportion would be ≥24% (80% CI±16%) using normal approximation methods. Sample size was later increased to better evaluate outcomes at the highest 400 mg bid dose. Sixty patients were enrolled such that ~15 patients completed 24 weeks of treatment and ≥10 patients completed 24 weeks of rilzabrutinib at a 400 mg bid starting dose.

Descriptive statistics and frequency tabulations summarized data per dose and overall. The primary efficacy endpoint and binomial confidence intervals (CI) were analyzed by the Clopper-Pearson method. Kaplan-Meier was used for time-to-event estimates.

### Results

### Patient Characteristics:

The study was initiated on Mar. 22, 2018 (data cutoff May 4, 2021); these results include all 60 patients who enrolled in the study and subsequently had the option to continue in the long-term extension (FIG. 1). Three patients completing the original protocol for 12 weeks of treatment are also included. Patients had a median age of 50 years (range, 19-74) and 57% were female (Table 1). Median baseline platelet counts were 15×10⁹/L (range, 2×10⁹/L−33×10⁹/L). Median duration of immune thrombocytopenia was 6.3 years (range, 0.4-52.5), and patients received a median of 4 prior unique immune thrombocytopenia treatments (range, 1-17). The most common prior therapies were glucocorticoids (92%), TPO-RAs including eltrombopag or romiplostim (58%), IVIG (43%), and rituximab (40%); 25% of patients had a prior splenectomy. Patients receiving glucocorticoids/TPO-RAs at enrollment with inadequate platelet counts (<30×10⁹/L) were allowed to continue stable doses of these medications.

TABLE 1
Characteristics of Patients at Baseline (Safety Population).

| | | | Initiated | |
|---|---|---|---|---|
| | | | 400 mg bid | |
| Characteristic | Overall (N = 60) | | (n = 45) | |
| Median age, y (range) | 50 | (19-74) | 49 | (19-74) |
| Sex-no. (%) | | | | |
| Male | 26 | (43) | 18 | (40) |
| Female | 34 | (57) | 27 | (60) |
| Median baseline platelet | 15 | (2-33) | 15 | (2-33) |
| count, x10⁹/L (range) | | | | |
| Median duration of ITP*, | 6.3 | (0.4-52.5) | 6.1 | (0.4-52.5) |
| years (range) | | | | |
| Median number of unique | 4 | (1-17) | 4 | (1-17) |
| prior ITP therapies^{†} (range) | | | | |
| Prior splenectomy^{†}-no. (%) | 15 | (25) | 11 | (24) |
| Most common prior ITP | | | | |
| therapies^{†-}no. (%) | | | | |
| Glucocorticoids | 55 | (92) | 42 | (93) |
| TPO-RAS | 35 | (58) | 24 | (53) |
| IVIG | 26 | (43) | 21 | (47) |
| Rituximab | 24 | (40) | 22 | (49) |
| Fostamatinib | 8 | (13) | 7 | (16) |

*Duration of disease is the difference between the date of first rilzabrutinib dose and the date of initial diagnosis.
^{†}Unique ITP therapies are identified using CMDECOD (the standardized or dictionary-derived text description of CMTRT or CMMODIFY), and splenectomy may be counted as one prior ITP therapy. Stable concomitant glucocorticoid/TPO-RA was defined as ≤10% dosing change within the 2 weeks prior to rilzabrutinib initiation as was allowed per protocol.

### Treatment:

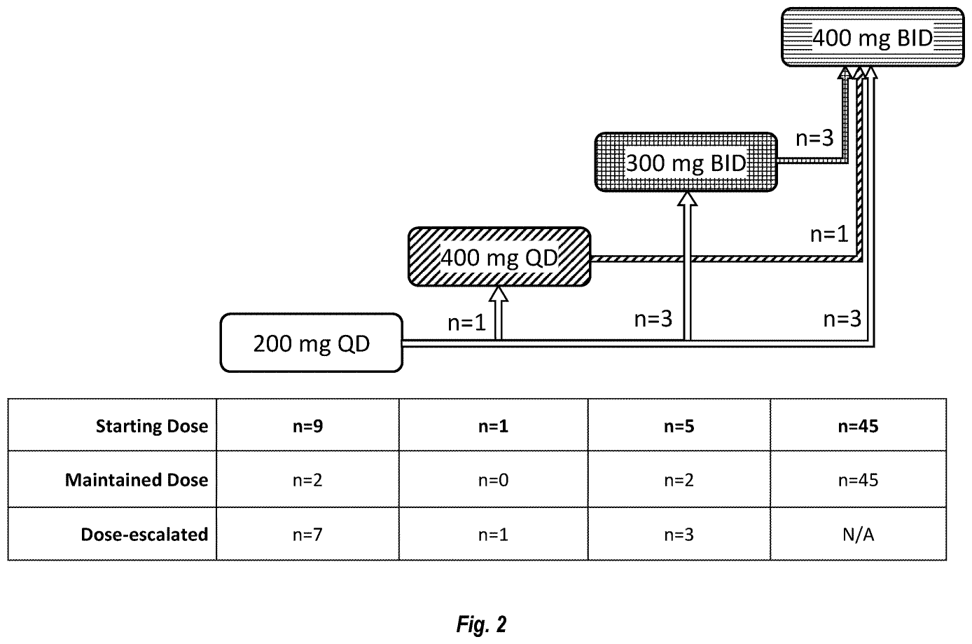

Patients received rilzabrutinib for a median of 167.5 days (range, 4-293). Nine patients initiated treatment at 200 mg qd; of these, 2 patients remained at the 200 mg qd dose, 1 patient dose-escalated to a maximum 400 mg qd, 3 patients to a maximum 300 mg bid (600 mg/day), and 3 patients to a maximum 400 mg bid (800 mg/day; n=3) over the treatment period (FIG. 2). One patient initiating rilzabrutinib at 400 mg qd dose-escalated to a maximum of 400 mg bid. Of five patients initiating 300 mg bid (600 mg/day), three escalated to a maximum dose of 400 mg bid. All 45 patients initiating 400 mg bid remained at that dose with no dose reductions/interruptions due to adverse events and no dose-limiting toxicities.

Overall, 40 patients received concomitant medication (not including rescue medication). The most frequently used were TPO-RAs in 24 patients and glucocorticoids in 23 patients. Out of 24 patients who achieved the primary efficacy endpoint, 4 patients received only concomitant TPO-RAs, 8 only glucocorticoids, 3 both, and 9 had none. Importantly, these patients were considered inadequate responders at baseline.

### Safety:

Rescue medication was used in seven (120) patients (1 patient at 200 mg qd, 1 at 300 mg bid, 5 at 400 mg bid; Table 2). Use of rescue medication was the cause of study discontinuation (as required by the study protocol) in four patients. Two additional patients received rescue medication but discontinued due to unrelated adverse events. One patient received rescue medication after discontinuing from the study due to lack of response.

TABLE 2
Patients Who Received Rescue Medication(s) and Information on
Their Discontinuations Due to Adverse Events, Lack of Response, and/or Rescue
Medication Use During the Study.

| | | | | | Time |
|---|---|---|---|---|---|
| | | Platelet | | | From |
| | | Count | Discontinuation | | Initial |
| | Baseline | Prior to | and/or Reason | | Treatment |
| Rilzabrutinib | Platelet | Rescue | for Rescue | Rescue | to Rescue |
| Dose* | Count | Medication | Medication | Medication | Use |
| 400 mg bid | 18 × 10⁹/L | 37 × 10⁹/L | Discontinued due | IVIG (iv) | Day 11 |
| | | (C1D8) | to rescue treatment | | |
| | | | use | | |
| 300 mg bid | 3 × 10⁹/L | 14 × 10⁹/L | Discontinued due | Methylprednisolone | Day 29 |
| | | (C2D1) | to rescue treatment | (250 mg qd iv) | |
| | | | use, treatment of | IVIG (70 g iv) | |
| | | | unrelated SAE | TPO-RA | |
| | | | (grade 3. | (eltrombopag 75 g | |
| | | | hematoma | qd oral) | |
| | | | following head | Platelet transfusion | |
| | | | contusion due to a | (2 U iv) | |
| | | | fall) | | |
| 200 mg qd | 24 × 10⁹/L | 4 × 10⁹/L | Discontinued due | Immunoglobulins | Day 36 |
| | | (C2D8) | to rescue treatment | NOS (30000 mg iv) | |
| | | | use | | |
| 400 mg bid | 13 × 10⁹/L | 15 × 10⁹/L | Discontinued due | Romiplostim 4 | Day 80 |
| | | (C3D22) | to rescue treatment | ug/wk sc | |
| | | | use | | |
| 400 mg bid | 16 × 10⁹/L | 3 × 10⁹/L | Treatment of | Methylprednisolone | Day 37 |
| | | (C2D8) | unrelated SAE | (125 and 80 mg/d | |
| | | | (grade 2 rectal | iv) | |
| | | | bleeding) | | |
| 400 mg bid | 17 × 10⁹/L, | 8 × 10⁹/1 | Treatment of | Methylprednisolone | Day 92 |
| | | (C4D8) | unrelated SAE | (125 mg qd iv), | |
| | | | (grade 4 | Prednisone (80 mg | |
| | | | thrombocytopenia) | po) | |
| | | | | IVIG ( 130 g gd iv) | |
| 400 mg bid | 21 × 10⁹/L | 16 × 10⁹/L | Lack of response | Anti-D | Day 90 |
| | | (C4D1) | to the study drug | immunoglobulin | |
| | | | | (3750 ug iv) | |

*Rilzabrutinib dose at the time of rescue medication use.

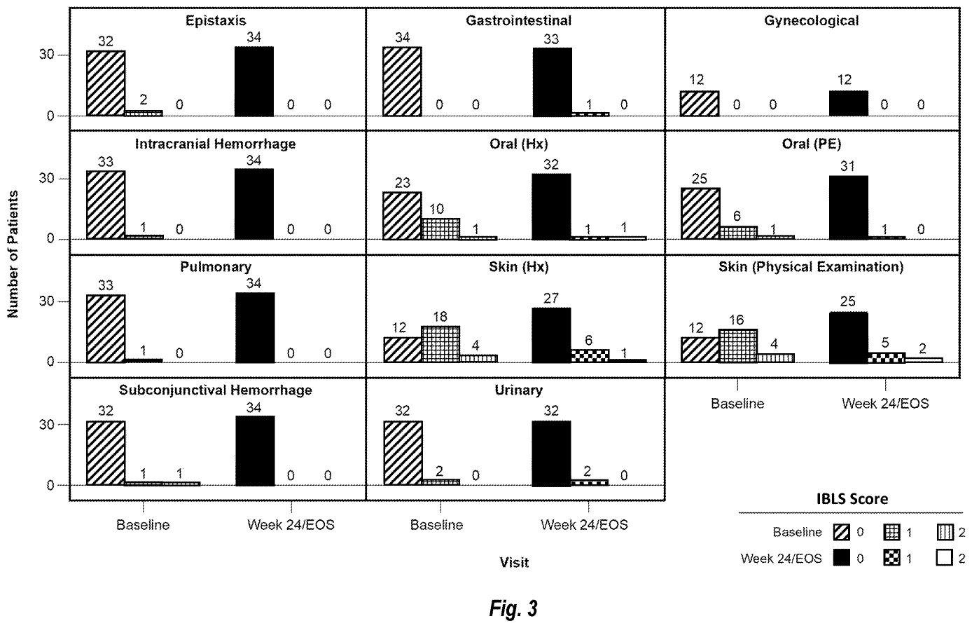

Thirty-one (52%) patients experienced ≥1 treatment-related, treatment-emergent adverse event; all were grade 1 or 2 and transient (Table 3; Table 4). The most common treatment-related, any-grade treatment-emergent adverse events were diarrhea (32%), nausea (30%), and fatigue (10%). One patient had a treatment-related grade 1 contusion (bleeding) and 1 patient had treatment-related grade 2 erysipelas (infection) that resolved on treatment. No treatment-related grade≥3 or serious adverse events were observed. There were no signs or symptoms of adverse events typically associated with BTK inhibitors (i.e., neutropenia, treatment-related infections, bleeding, thrombotic events, fungal infections, or atrial fibrillation). There was 1 patient death unrelated to treatment; this patient discontinued rilzabrutinib 400 mg bid on day 8 due to exacerbation of pre-existing Evans syndrome and died 135 days after initiating the study. The IBLS bleeding scale scores within each domain showed no increase in bleeding from baseline through the end of treatment, but instead improved at skin and oral sites (FIG. 3).

TABLE 3
Summary of Treatment-Emergent Adverse Events by Grade
(Treatment-Related Events Occurring in >5%; Safety Population; N=60).

| | TEAEs Due to Any Cause | | | | Treatment-Related TEAEs* | | | |
|---|---|---|---|---|---|---|---|---|
| | Any | Grade | Grade | Grade | Any | Grade | Grade | Grade |
| Event | Grade | 1 | 2 | 3/4 | Grade | 1 | 2 | 3/4 |
| | number of patients (percent) | | | | | | | |
| Any TEAE | 48 (80) | 43 (72) | 30 (50) | 8 | 31 (52) | 27 (45) | 15 (25) | — |
| | | | | (13)† | | | | |
| Diarrhea | 22 (37) | 19 (32) | 3 (5) | — | 19 (32) | 16 (27) | 3 (5) | — |
| Nausea | 21 (35) | 18 (30) | 3 (5) | — | 18 (30) | 16 (27) | 2 (3) | — |
| Fatigue | 12 (20) | 10 (17) | 2 (3) | — | 6 (10) | 5 (8) | 1 (2) | — |
| Abdominal | 6 (10) | 6 (10) | — | — | 4 (7) | 4 (7) | — | — |
| distension | | | | | | | | |
| Vomiting | 4 (7) | 3 (5) | 1 (2) | — | 3 (5) | 2 (3) | 1 (2) | — |

- represents zero events
*Treatment-related TEAEs were all grade 1 or 2; none were grade ≥3.
†Eight patients experienced grade 3/4 TEAEs (multiple may occur in a single patient) due to any cause and unrelated to rilzabrutinib treatment including grade 3 anemia (n = 2); one each with grade 3 abnormal alanine aminotransferase levels, contusion, gastrointestinal hemorrhage, hematoma, immune thrombocytopenia, myelofibrosis, or thrombocytopenia; and one each with grade 4 Evans syndrome or thrombocytopenia.

TABLE 4
Summary of Treatment-Emergent Adverse Events by Grade
(Occurring in >2 Patients Irrespective of Cause or ≥1 Patient
Treatment-Related; Safety Population; N=60).

| | TEAEs Due to Any Cause | | | | Treatment-Related TEAEs* | | | |
|---|---|---|---|---|---|---|---|---|
| | Any | Grade | Grade | Grade | Any | Grade | Grade | Grade |
| | grade | 1 | 2 | 3/4 | grad | 1 | 2 | 3/4 |
| | number of patients (percent) | | | | | | | |
| Any TEAE | 48 (80) | 43 (72) | 30 (50) | 8 (13) | 31 (52) | 27 (45) | 15 (25) | 0 (0) |
| Gastrointestinal | | | | | | | | |
| Diarrhea | 22 (37) | 19 (32) | 3 (5) | — | 19 (32) | 16 (27) | 3 (5) | — |
| Nausea | 21 (35) | 18 (30) | 3 (5) | — | 18 (30) | 16 (27) | 2 (3) | — |
| Abdominal | 6 (10) | 6 (10) | — | — | 4 (7) | 4 (7) | — | — |
| distension | | | | | | | | |
| Dyspepsia | 4 (7) | 3 (5) | 1 (2) | — | 1 (2) | 1 (2) | — | — |
| Vomiting | 4 (7) | 3 (5) | 1 (2) | — | 3 (5) | 2 (3) | 1 (2) | — |
| Abdominal pain | 3 (5) | 1 (2) | 2 (3) | — | 2 (3) | — | 2 (3) | — |
| Gingival bleeding | 3 (5) | 3 (5) | | | | | | |
| Upper abdominal | 3 (5) | 2 (3) | 1 (2) | — | 2 (3) | 1 (2) | 1 (2) | — |
| pain | | | | | | | | |
| Abdominal | 1 (2) | 1 (2) | — | — | 1 (2) | 1 (2) | — | — |
| discomfort | | | | | | | | |
| Defecation | 1 (2) | — | 1 (2) | — | 1 (2) | — | 1 (2) | — |
| urgency | | | | | | | | |
| Epigastric | 1 (2) | — | 1 (2) | — | 1 (2) | — | 1 (2) | — |
| discomfort | | | | | | | | |
| Frequent bowel | 1 (2) | — | 1 (2) | — | 1 (2) | — | 1 (2) | — |
| movements | | | | | | | | |
| General disorders | | | | | | | | |
| and administrative | | | | | | | | |
| site conditions | | | | | | | | |
| Fatigue | 12 (20) | 10 (17) | 2 (3) | — | 6 (10) | 5 (8) | 1 (2) | — |
| Peripheral | 3 (5) | 3 (5) | — | — | — | — | — | — |
| swelling | | | | | | | | |
| Edema | 2 (3) | 2 (3) | — | — | 1 (2) | 1 (2) | — | — |
| Malaise | 1 (2) | 1 (2) | — | — | 1 (2) | 1 (2) | — | — |
| Musculoskeletal | | | | | | | | |
| and connective | | | | | | | | |
| tissue disorders | | | | | | | | |
| Back pain | 8 (13) | 7 (12) | 1 (2) | — | 1 (2) | — | 1 (2) | — |
| Arthralgia | 5 (8) | 3 (5) | 2 (3) | — | — | — | — | — |
| Costochondritis | 2 (3) | 2 (3) | — | — | — | — | — | — |
| Muscle spasms | 2 (3) | 2 (3) | — | — | 1 (2) | 1 (2) | — | — |
| Myalgia | 1 (2) | 1 (2) | — | — | 1 (2) | 1 (2) | — | — |
| Infections and | | | | | | | | |
| Infestations* | | | | | | | | |
| Nasopharyngitis | 3 (5) | 2 (3) | 1 (2) | — | — | — | — | — |
| Upper respiratory | 3 (5) | 2 (3) | 1 (2) | — | — | — | — | — |
| tract infection | | | | | | | | |
| Urinary tract | 2 (3) | 1 (2) | 1 (2) | — | — | — | — | — |
| infection | | | | | | | | |
| Erysipelas | 1 (2) | — | 1 (2) | — | 1 (2) | — | 1 (2) | — |
| Nervous system | | | | | | | | |
| disorders | | | | | | | | |
| Headache | 7 (12) | 7 (12) | — | — | 2 (3) | 2 (3) | — | — |
| Dizziness | 2 (3) | 2 (3) | — | — | — | — | — | — |
| Akathisia | 1 (2) | 1 (2) | — | — | 1 (2) | 1 (2) | — | — |
| Hypoesthesia | 1 (2) | — | 1 (2) | — | 1 (2) | — | 1 (2) | — |
| Respiratory, | | | | | | | | |
| thoracic, and | | | | | | | | |
| mediastinal | | | | | | | | |
| disorders | | | | | | | | |
| Cough | 2 (3) | 2 (3) | — | — | — | — | — | — |
| Dyspnea | 2 (3) | 2 (3) | — | — | — | — | — | — |
| Dyspnea exertional | 2 (3) | 2 (3) | — | — | — | — | — | — |
| Epistaxis | 2 (3) | 2 (3) | — | — | — | — | — | — |
| Nasal congestion | 2 (3) | 1 (2) | 1 (2) | — | — | — | — | — |
| Skin and | | | | | | | | |
| subcutaneous | | | | | | | | |
| tissue disorders | | | | | | | | |
| Petechiae | 4 (7) | 4 (7) | — | — | — | — | — | — |
| Rash | 3 (5) | 1 (2) | 2 (3) | — | 1 (2) | 1 (2) | — | — |
| Alopecia | 2 (3) | 2 (3) | — | — | — | — | — | — |
| Purpura | 2 (3) | 2 (3) | — | — | — | — | — | — |
| Pruritus | 2 (3) | 1 (2) | 1 (2) | — | 2 (3) | 1 (2) | 1 (2) | — |
| Maculo-papular | 1 (2) | 1 (2) | — | — | 1 (2) | 1 (2) | — | — |
| rash | | | | | | | | |
| Blood and | | | | | | | | |
| lymphatic system | | | | | | | | |
| disorders | | | | | | | | |
| Anemia | 4 (7) | — | 2 (3) | 2 (3) | — | — | — | — |
| Thrombocytopena | 2 (3) | — | — | 2 (3) | — | — | — | — |
| Metabolism and | | | | | | | | |
| nutrition disorders | | | | | | | | |
| Hypophosphatemia | 2 (3) | — | 2 (3) | — | — | — | — | — |
| Decreased | 1 (2) | 1 (2) | — | — | 1 (2) | 1 (2) | — | — |
| appetite | | | | | | | | |
| Ilypokalemia | 1 (2) | 1 (2) | — | — | 1 (2) | 1 (2) | — | — |
| Other | | | | | | | | |
| Contusion | 11 (18) | 7 (12) | 3 (5) | 1 (2) | 1 (2) | 1 (2) | — | — |
| Arthropod bite | 3 (5) | 3 (5) | — | — | — | — | — | — |
| Conjunctival | 5 (8) | 5 (8) | — | — | — | — | — | — |
| hemorrhage | | | | | | | | |
| Heavy menstrual | 2 (3) | 1 (2) | 1 (2) | — | — | — | — | — |
| bleeding | | | | | | | | |
| Abnormal dreams | 1 (2) | 1 (2) | — | — | 1 (2) | 1 (2) | — | — |
| Micturition | 1 (2) | 1 (2) | — | — | 1 (2) | 1 (2) | — | — |
| urgency | | | | | | | | |
| I lot flush | 1 (2) | — | 1 (2) | — | 1 (2) | — | 1 (2) | — |
| Increased white | 1 (2) | — | 1 (2) | — | 1 (2) | — | 1 (2) | — |
| blood cell count | | | | | | | | |

— represents zero events;
TEAE, treatment-emergent adverse event.
*Treatment-related TEAEs were all grade 1 or 2; none were grade ≥3.
†Eight patients experienced grade 3/4 TEAEs (multiple may occur in a single patient) due to any cause and unrelated to rilzabrutinib treatment including grade 3 anemia (n = 2); one each with grade 3 abnormal alanine aminotransferase levels, contusion, gastrointestinal hemorrhage, hematoma, immune thrombocytopenia, myelofibrosis, or thrombocytopenia; and one each with grade 4 Evans syndrome or thrombocytopenia.
‡Other infections and infestations not listed in the table that were unrelated to treatment and that occurred in a single patient each included grade 1 candida infection, fungal skin infection, and osteomyelitis; and grade 2 infected skin ulcer and oral herpes.

Seven patients experienced a TEAL that resulted in study drug discontinuation (4 (70%) patients with gastrointestinal (GI) disorders, 2 (40%) with lymphatic system disorders, and 1 (2%) with administrative and site conditions). All were unrelated to rilzabrutinib treatment, with the exception of grade 1 fatigue in 1 patient and grade 2 GI disorders in 2 patients. All TEAEs leading to study drug discontinuation were of grade 1 or 2 severity, with the exception of grade 3 GI hemorrhage, grade 4 thrombocytopenia, and grade 4 Evans syndrome in 1 patient each. Four of the TEAEs leading to study drug discontinuation were reported as serious adverse events (grade 2 rectal hemorrhage, grade 3 GI hemorrhage, grade 4 thrombocytopenia, and grade 4 Evans syndrome); none were related to rilzabrutinib treatment.

### Efficacy:

All 60 patients were evaluable for efficacy for this 24-week study. The primary endpoint (≥2 consecutive platelet counts≥50×10⁹/L and increased ≥20×10⁹/L from baseline without requiring rescue medication) was achieved in 24 patients (400; 9500 CI, 28%-53%) (Table 5). According to dose level at any time during the study (patients could respond at more than one dose level), 1 of 9 patients (11) achieved the primary endpoint at 200 mg qd, 2/8 (25%) at 400 mg qd, 4/12 (33%) at 300 mg bid, and 20/52 (38%) at 400 mg bid. In 45 patients who initiated and maintained rilzabrutinib at a dose of 400 mg bid throughout the study, 18 (40) patients achieved the primary platelet response (Table 5 and Table 6).

TABLE 5
Efficacy Outcomes.

| | All Patients | | Initiated 400 mg | |
|---|---|---|---|---|
| | (N = 60) | | bid (n = 45) | |
| Primary endpoint*, n (%) [95% CI] | 24 | (40) | 18 | (40) |
| Secondary efficacy endpoints | [28, 53] | | [26, 56] | |
| Percent of weeks with platelet counts | | | | |
| ≥50 × 10⁹/L, mean (SD) | | | | |
| All patients | 29 | (35) | 28 | (36) |
| Responders meeting primary endpoint*^{‡} | n = 24 | | n = 18 | |
| | 65 | (25) | 67 | (26) |
| Proportion of patients with ≥4 out of the | | | | |
| final 8 platelet counts ≥50 × 10⁹/L, n (%) | | | | |
| [95% CI] | | | | |
| All patients | 17 | (28) | 14 | (31) |
| | [17, 41] | | [18, 47] | |
| Responders meeting primary endpoint*^{‡} | n = 24 | | n = 18 | |
| | 17 | (71) | 14 | (78) |
| Mean change (SD) from baseline to the | [49, 87] | | [52, 94] | |
| average of post day 1 platelet counts for | | | | |
| patients with >4 weeks of treatment, ×10⁹/L | | | | |
| All patients (with >4 weeks treatment) | 29 | (40) | 31 | (43) |
| Responders meeting primary endpoint*^{‡} | n = 24 | | n = 18 | |
| | 58 | (43) | 64 | (48) |
| Number of weeks with platelet counts | | | | |
| ≥50 × 10⁹/L, median (range) | | | | |
| All patients | 1 | (0-26) | 0 | (0-24) |
| Responders meeting primary endpoint*^{‡} | n = 24 | | n = 18 | |
| | 16 | (2-26) | 14 | (3-24) |
| Number of weeks with platelet counts | | | | |
| ≥30 × 10⁹/L, median (range) | | | | |
| All patients | 5 | (0-32) | 5 | (0-24) |
| Responders meeting primary endpoint*^{‡} | n = 24 | | n = 18 | |
| | 21 | (3-32) | 21 | (7-24) |
| Time to first platelet count ≥50 × 10⁹/L, | | | | |
| median number of days (range) | | | | |
| All patients | 11.5 | (7-142) | 12.5 | (8-142) |
| Responders meeting primary endpoint*^{‡} | n = 24 | | n = 18 | |
| | 10.5 | (7-71) | 11.5 | (8-71) |

Note:
Efficacy endpoints by initial dose level of rilzabrutinib for the 24-week study (and prior to the long-term extension) are reported in Table 6.
CI, confidence interval (based on Wilson Score method). The widths of the confidence intervals have not been adjusted for multiplicity and should not be used to definitively infer efficacy.
*The primary endpoint for platelet response was defined as ≥2 consecutive platelet counts, separated by at least 5 days, of ≥50×109/L and increased ≥20 × 10⁹/L from baseline without requiring rescue medication in the 4 weeks prior to the latest elevated platelet count.
^{†}Included achievement of primary endpoint at rilzabrutinib doses of 200 mg qd (n = 1), 400 mg qd (n = 2), 300 mg bid (n = 4), and 400 mg bid (n = 20) at any time; primary endpoint may have been reached at different doses in the same patient.
‡Post-hoc analyses of responding patients.

TABLE 6
Efficacy Outcomes by Initial Dose of Rilzabrutinib.

| | 200 mg | 400 mg | 300 mg | 400 mg | All |
|---|---|---|---|---|---|
| Initial Dose of | qd | qd | bid | bid | Patients |
| Rilzabrutinib | (n = 9) | (n = 1) | (n = 5) | (n = 45) | (N = 60) |
| Primary endpoint*, n | 4 | 0 | 2 | 18 | 24 |
| (%) [95% CI] | (44) | (0) | (40) | (40) | (40) |
| Secondary efficacy | [14, 79] | [0, 98] | [5, 85] | [26, 56] | [28, 53] |
| endpoints | | | | | |
| Percent of weeks with | 28 | 0 | 40 | 28 | 29 |
| platelet counts | (29) | (NA) | (41) | (36) | (35) |
| ≥50 × 10⁹/L, mean (SD) | | | | | |
| Proportion of patients | 1 | 0 | 2 | 14 | 17 |
| with ≥4 out of the final | (11) | (0) | (40) | (31) | (28) |
| 8 platelet counts | [0.3, 48] | [0, 98] | [5, 85] | [18, 47] | [17, 41] |
| ≥50 × 10⁹/L, n (%) | | | | | |
| [95% CI] | | | | | |
| Mean change (SD) | n = 8 | n = 1 | n = 4 | n = 41 | n = 54 |
| from baseline to the | 19 | −0.4 | 30 | 31 | 29 |
| average of post day 1 | (24) | (NA) | (27) | (43) | (40) |
| platelet counts for | | | | | |
| patients with >4 weeks | | | | | |
| of treatment, ×10⁹/L | | | | | |
| Number of weeks with | 2.5 | 0 | 5 | 0 | 1 |
| platelet counts | (0-20) | (0-0) | (0-26) | (0-24) | (0-26) |
| ≥50 × 10⁹/L, median | | | | | |
| (range) | | | | | |
| Number of weeks with | 4 | 0 | 15 | 5 | 5 |
| platelet counts | (0-28) | (0-0) | (0-32) | (0-24) | (0-32) |
| ≥30 × 10⁹/L, median | | | | | |
| (range) | | | | | |
| Time to first platelet | 8 | — | 8 | 12.5 | 11.5 |
| count ≥50 × 10⁹/L, | (8-63) | | (7-8) | (8-142) | (7-142) |
| median number of days | | | | | |
| (range) | | | | | |

CI, confidence interval (based on Wilson Score method); NA, not applicable; NE, not evaluated. The widths of the confidence intervals have not been adjusted for multiplicity and should not be used to definitively infer efficacy.
*The primary endpoint for platelet response was defined as ≥2 consecutive platelet counts, separated by at least 5 days, of ≥50 × 10⁹/L and increased ≥20 × 10⁹/L from baseline without requiring rescue medication in the 4 weeks prior to the latest elevated platelet count.

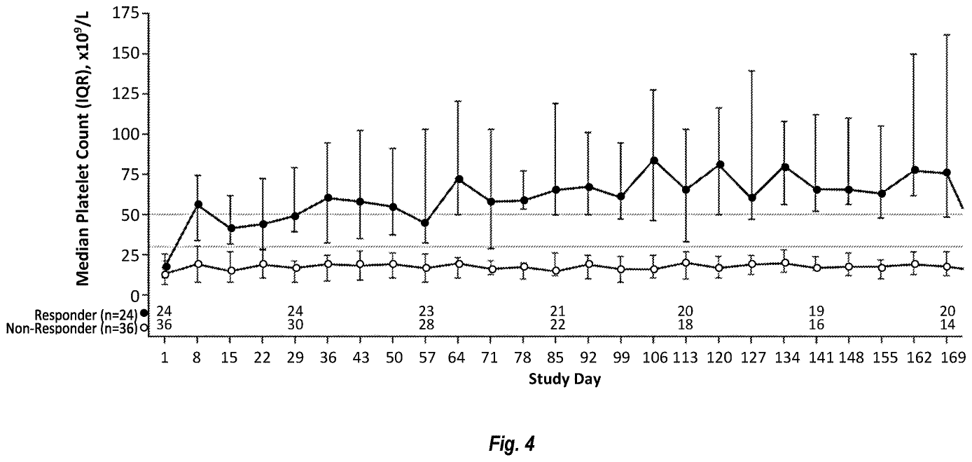

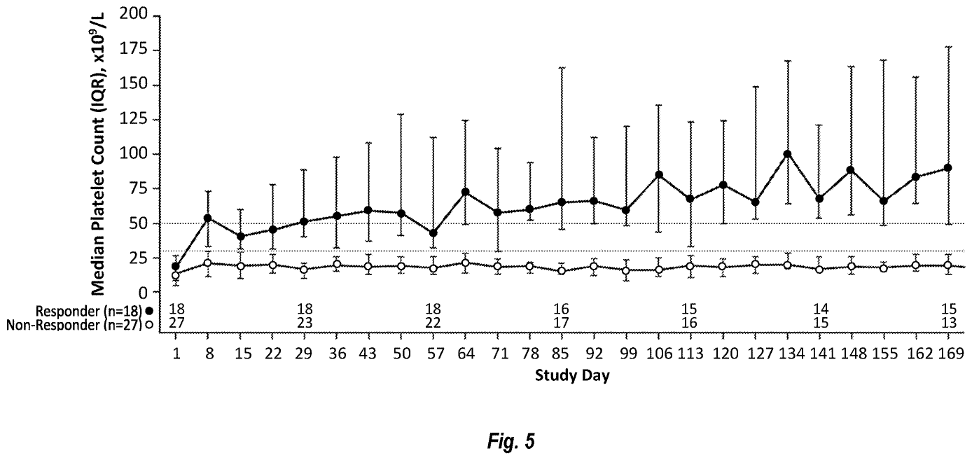

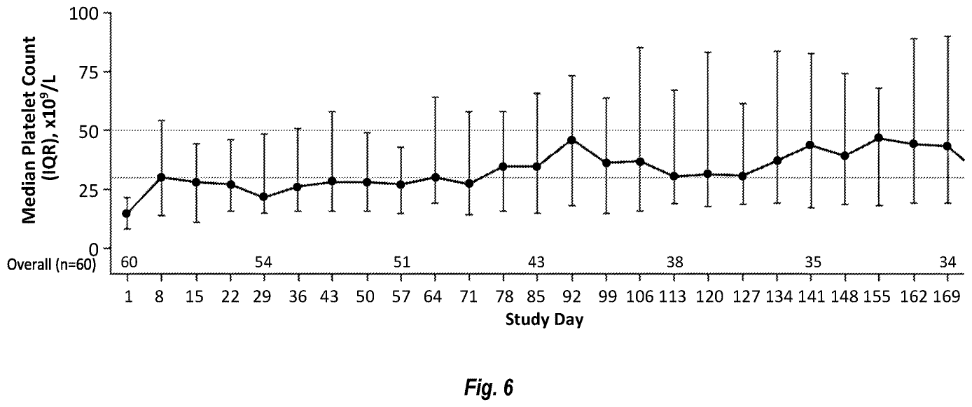

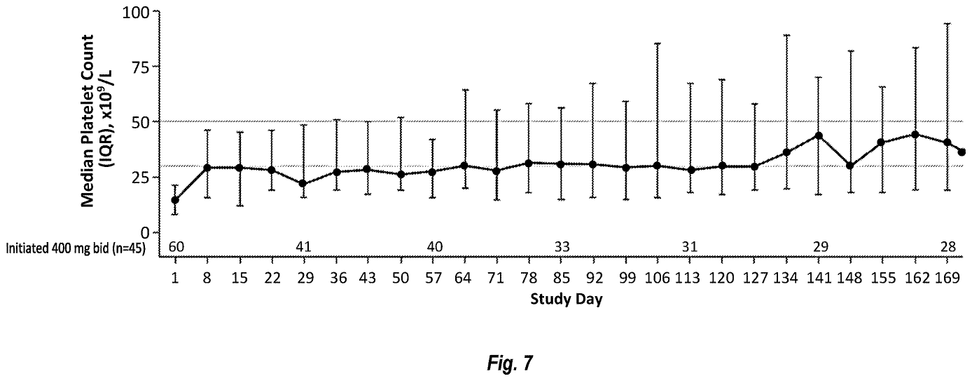

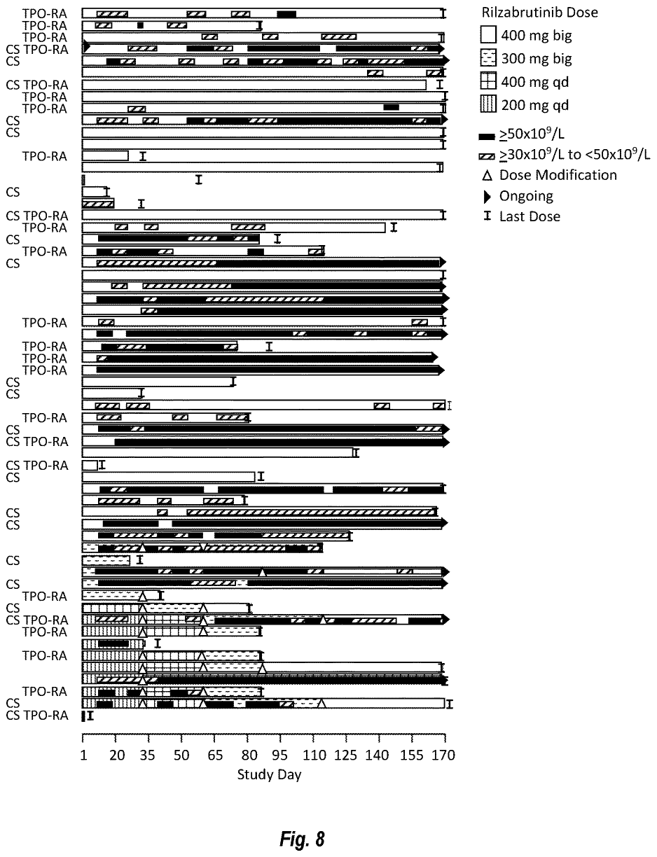

The mean percent of weeks with platelet counts≥50×10⁹/L was 29% overall; post-hoc analyses showed that the mean percent of weeks was 65% for responder patients meeting the primary endpoint. Based on a safety profile that was low-grade and consistent efficacy observed at the highest evaluated dose, the minimum effective dose for rilzabrutinib was identified as 400 mg bid. No escalations beyond 400 mg bid were tested. The proportion of patients with ≥4 of final 8 platelet counts≥50×10⁹/L was 17 (28%) overall and 14 (31%) for patients initiating 400 mg bid (Table 5). Mean changes from baseline to the average of post-day 1 platelet counts in patients completing >4 weeks of treatment were 29×10⁹/L (SD, 40×10⁹/L) for all patients and 31×10⁹/L (SD, 43×10⁹/L) for patients initiating 400 mg bid. Platelet counts of ≥50×10⁹/L and ≥30×10⁹/L were maintained for median 1 and 5 weeks, respectively, in the overall patient population. When evaluating responding patients overall (n=24) and those initiating 400 mg bid (n=18) who met the primary efficacy platelet counts, these respective groups maintained platelet counts of ≥50×10⁹/L for a median of 16 and 14 weeks, as well as platelet counts of ≥30×10⁹/L for a median of 21 weeks for both groups (FIGS. 4 and 5). The median time to first platelet count≥50×10⁹/L was 11.5 days for all patients and 12.5 days for those initiating 400 mg bid (FIGS. 6-8, Table 5, and Table 6).

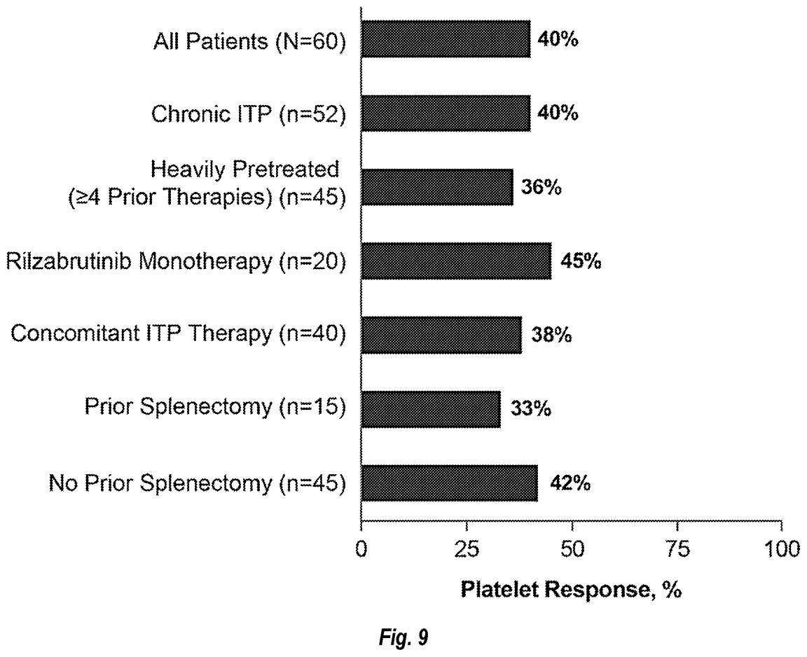

Primary platelet responses overall were comparable across subgroups of patients who had chronic immune thrombocytopenia (duration for >12 months) and persistent immune thrombocytopenia (duration of 3-12 months), were heavily pretreated with ≥4 prior immune thrombocytopenia therapies, had received either rilzabrutinib monotherapy, concomitant immune thrombocytopenia therapy, or prior splenectomy (FIG. 9).

This clinical study demonstrated that BTK inhibition with rilzabrutinib is effective at suppressing immune-mediated platelet destruction in immune thrombocytopenia, thus providing a novel mechanism for targeting underlying immune thrombocytopenia pathology. Patients had experienced immune thrombocytopenia for a median duration of 6.3 years with a median of four unique prior therapies. Notably, eligibility was based on an inability to maintain an adequate response to prior/concomitant therapies (including splenectomy) and ongoing need for treatment. Oral rilzabrutinib was associated with only low-grade toxicity across all doses, in alignment with early clinical studies in healthy volunteers (Smith P F et al., 2017) and projected doses from preclinical studies (Langrish C L et al., 2021). There was no evidence of infections, thrombotic events, cardiac arrhythmias, liver toxicity, or increased bleeding typically associated with BTK inhibitors and/or TPO-RAs though follow up time was limited (Aguilar C, 2018; Shatzel J J et al., 2017; Ghanima W et al., 2018; von Hundelshausen P and Seiss W, 2021). In addition, no patient developed platelet counts>400×10⁹/L. Rilzabrutinib's safety profile was consistent with prior phase 1 healthy volunteer results (Smith P F et al., 2017).

Rilzabrutinib led to rapid platelet responses and clinically significant platelet responses (≥50×10⁹/L) in 40% of rilzabrutinib-treated patients. Important treatment factors for immune thrombocytopenia patients are attaining rapid improvement and maintaining durable responses over time. Median time to first platelet count≥50×10⁹/L was rapid at 11.5 days, which was maintained in responders for a median of 72% of weeks in the 24-week treatment period. Importantly, platelet responses were consistent across baseline subgroups.

No standard treatment recommendations currently exist for immune thrombocytopenia patients with multiple relapses. Treatment/study comparisons are further complicated by a range of timepoints and platelet response endpoints. Recent ASH treatment guidelines for immune thrombocytopenia lasting ≥3 months recommend TPO-RAs, rituximab, or splenectomy (Neunert et al., 2019). Immunomodulators for relapsed immune thrombocytopenia include spleen tyrosine kinase inhibitor fostamatinib (Tavalisse (fostamatinib) prescribing information, 2020) and anti-CD20 antibody rituximab (Ghanima W et al., 2015; Deshayes S et al., 2019; Tjonnfjord E et al., 2020). Although durable remissions are high with splenectomy (~60-70% of patients), it is generally viewed less favorably due to potential surgical complications, higher risk for infections/thromboembolic events, and lifelong increased risk of sepsis with capsuled microorganisms (Cooper N and Ghanima W, 2019; Kojouri K et al., 2004). Treatment choice is generally individualized based on patient age/characteristics, comorbidities, disease duration, bleeding frequency, treatment availability, and patient preferences (Neunert et al., 2019). Despite significant advances in immune thrombocytopenia therapeutic options over the last 15 years, a subset of patients remains refractory to existing therapies and durable remission remains elusive. Because the patients here were heavily pretreated and many were viewed by investigators as having limited treatment options, inhibition of BTK by rilzabrutinib provided a novel therapeutic approach by targeting immune-mediated platelet destruction and impaired platelet production.

This phase 1-2, dose-finding clinical study established a role for BTK inhibition in immune thrombocytopenia treatment. Overall, oral rilzabrutinib demonstrated rapid and durable clinical activity in up to half of immune thrombocytopenia patients following multiple prior therapies and had a low-grade safety profile. The rilzabrutinib 400 mg bid dose was shown as safe and effective.

### Example 2: Cognitive Impairment Among Patients with Chronic Immune Thrombocytopenia

Participants (N=60) in the ongoing, global phase 1/2 study (NCT03395210) assessing the safety and efficacy of the Bruton tyrosine kinase inhibitor rilzabrutinib in ITP, were eligible for cognitive testing at the pre-treatment baseline assessment (the evaluation prior to rilzabrutinib treatment). Cognition was evaluated using tests from the Cogstate Brief Battery (CBB); the detection test (DET), identification test (IDN), one card learning test (OCL), and one back test (ONB) were used to measure psychomotor function, attention, visual learning, working memory, respectively (Table 7). The individual and composite scores from these tests have a high sensitivity to cognitive dysfunction in clinical contexts (Hinton-Bayre A D, 2010; Maruff P et al., 2009).

The CBB test was presented to all patients by trained and certified raters using a laptop or tablet computer according to standardized administration procedures. At the beginning of each test, written instructions and testing rules were presented on the screen and read aloud to the patient. An interactive demonstration of the CBB followed and practice trials were presented to allow patients to demonstrate their understanding of the rules. Each CBB test was presented in the form of a card game and required patients to respond using a “yes” or “no” button on each trial, with “yes” responses made with the right hand. Patients were instructed and trained to “respond as fast and as accurately as possible.” For each test, responses faster than 100 milliseconds (msec) were defined as anticipatory and responses slower than 3000 msec were defined as abnormally slow, with both excluded from further analysis. For each test, the proportion of correct responses and the average speed of correct responses were computed automatically. To optimize performance data for use in parametric statistical analyses, distributions of proportional correct scores were transformed using an arcsine square root transformation (arcsine), and distributions of mean reaction times were transformed using a logarithmic base 10 (log 10 msec) transformation (Maruff et al. 2009). The CBB tests are described below in their order of presentation.

The detection (DET) test assessed psychomotor function and was based on the simple reaction time test paradigm. Patients were to attend to the task in the center of the screen and follow the rule, “Has the card turned face up? Press ‘yes’ as soon as it does.” The task continued until 25 correct responses were recorded or the maximum allowed time (2 min) had elapsed. The outcome measure for this test was speed of performance.

The identification (IDN) test assessed visual attention and was based on a choice reaction time test paradigm. Patients were to attend to the card in the center of the screen and follow the rule “Is the face-up card red?” as the card turned, pressing the “yes” or “no” buttons. This task continued until 35 correct responses were recorded or the maximum allowed time (2 min) had elapsed. The outcome measure for this test was speed of performance.

The one card learning (OCL) test assessed visual learning and was based on a pattern separation paradigm. Patients were to attend to the card in the center of the screen and follow the rule “Have you seen this card before in this task?” as the card turned, pressing “yes” or “no.” Patients were required to learn a series of four cards (target cards) presented in pseudo-random order with 6 distracter cards (i.e., non-repeating) in groups of 10 trials for 80 trials or until the maximum time allowed (3 min) had elapsed. The outcome measure for this test was accuracy of performance.

The one back (ONB) test assessed working memory and was based on the n-back (1-back condition) paradigm. Patients were to attend to the card in the center of the screen and use the rule “Is this card the same as that on the immediately previous trial?” as the card turned over, pressing “yes” or “no.” Forty-two cards were shown in which correct responses occurred on 50% of trials. This task continued until the 42 trials were completed or the maximum allowed time (3 min) had elapsed. The outcome measure for this test was accuracy of performance.

TABLE 7
Outcome Measures of Cogstate Tests.

| | | | | |
|---|---|---|---|---|
| | Main | | | Direction of |
| | Cognitive | | Primary | Scores for |
| Test or | Function | Cognitive | Performance | Better |
| Composite | Measured | Domain | Measure | Performance |

Individual Tests

| | | | | |
|---|---|---|---|---|
| Detection Test | Psychomotor | Attention | Speed of performance; | Lower score = |
| (DET)* | function | | mean of the log10 | better |
| | | | transformed reaction | performance |
| | | | times for correct | |
| | | | responses | |
| Identification | Attention | Attention | Speed of performance, | Lower score |
| Test (IDN)* | | | mean of the log10 | better |
| | | | transformed reaction | performance |
| | | | times for correct | |
| | | | responses | |
| One Card | Visual | Memory | Accuracy of | Higher score = |
| Learning Test | learning | and | performance; arcsine | better |
| (OCL) | | learning | transformation of the | performance |
| | | | square root of the | |
| | | | proportion of correct | |
| | | | responses | |
| One Back Test | Working | Executive | Accuracy of | Higher score |
| (ONB) | memory | function | performance, arcsine | better |
| | | | transformation of the | performance |
| | | | square root of the | |
| | | | proportion of correct | |
| | | | responses | |

Composite Scores

| | | | | |
|---|---|---|---|---|
| Psychomotor | Rapid and | Attention | Combination of age | Higher score |
| Function/ | automatic | | standardized scores on | better |
| Attention | decisions | | the DET and IDN, re- | performance |
| | | | standardized against | |
| | | | population norms | |
| Learning/ | Accurate and | Higher | Combination of age | Higher score = |
| Working | careful | cognitive | standardized scores on | better |
| Memory | decision | function | the OCL and ONB, re- | performance |
| | making | | standardized against | |
| | | | population norms | |

*Indicates the sign of standardized scores is reversed so that negative standard scores indicate performance below normal mean (and vice versa).

Analysis of performance on the cognitive tests occurred in three stages. For the first stage, each participant's score on the main outcome measure for each of the four cognitive tests was expressed as a standardized Z-score using the age-stratified (5-year strata) mean and standard deviation from the CBB normative data (Bloch et al. 2016; Collie et al. 2003; Maruff et al. 2009; Maruff et al. 2013). Z-scores were used to measure the distance between an observed value and a reference mean using standard deviation units (SD) and could be positive or negative, indicating that the observed value lay above or below the mean, respectively. Z-scores≤−1 denoted clinically important impairment, and (for tests that used performance speed as the outcome, the sign of standardized scores was reversed, so that all standardized scores with lower values indicated performance worse than normal and vice versa.).

For each participant, the standardized scores for the detection and identification tests were averaged to provide a psychomotor function/attention composite score. Additionally, standardized scores for the one card learning and one back tests were averaged to provide a learning/working memory composite score. The magnitude of impairment in cognition in the ITP group was then determined by computing group means and 95% confidence intervals (CI) for each individual and composite score. A clinically important cognitive impairment was defined as performance on either of the composite scores that was less than or equal to −1 (i.e., ≤−1). Differences were considered statistically significant if the 95% CIs did not include zero.

The second stage of the analysis determined the number and percentage of patients with ITP who showed clinically important impairment on the individual tests and psychomotor function/attention and learning/working memory composite scores. The composite scores were used for these analyses because of greater reliability than the individual participant tests from which they were derived (Maruff et al. 2013).

For the third stage, the participants were classified into subgroups based on the presence or absence of cognitive impairment on each of the composite scores. To explore the extent to which the baseline factors of ITP were associated with a classification of cognitive impairment, data for the demographic and clinical characteristics were compared between subgroups. Analyses were based on two-sample t-test assuming unequal variances. Mean difference values between groups <0 signified greater cognitive impairment.

Performance on each test was standardized relative to age-matched normative data (Bloch et al. 2016; Collie et al. 2003; Maruff et al. 2009; Maruff et al. 2013), and the score was converted to a Z-score by subtracting the age-matched mean from the Cogstate normative data sample and divided by the age-matched standard deviation from the same normative sample. The multiplicand was 1 for the one card learning test, where a higher score indicated better cognitive performance, and −1 for detection, identification, and one back tests, where a lower score indicated better cognitive performance. To increase reliability of performance estimates, Z-scores from two baseline visits were averaged.

### Results

Cognitive testing with evaluable scores was available from baseline assessments (i.e., prior to rilzabrutinib treatment) for 49 of 60 enrolled patients (82%) (Table 8). At baseline, the 49 patients with CBB assessments had a mean age of 50 years (SD of 13 years), 57% were female, and 71% had an ITP duration of ≥3 years. Prior ITP therapies included corticosteroids (n=47), TPO-RAs (n=38), and splenectomy (n=14). 14 patients each used concomitant corticosteroids only or thrombopoietin receptor agonists (TPO-RAs) only, and 7 patients used both concomitant therapies. Medical history included diabetes (n=7), hypertension (n=17), depression/anxiety (n=8), and stroke/CNS bleed (n=1).

Cognitive impairment, defined as having two or more of the detection, identification, one card learning, and one back tests with a score≤−1, was observed in 29 of 49 (59%) patients with ITP (Table 8). According to baseline factors, only the use of concomitant TPO-RAs was identified as showing significantly more patients with impairment than those not receiving concomitant TPO-RAs (p=0.0450).

TABLE 8
Baseline Demographics and Characteristics Overall and
Comparison of Factors in Patients with or without Cognitive Impairment.

| | Patients | Impaired Patients* | |
|---|---|---|---|
| Baseline Characteristics | N (%) | n/N (%) | P-value^{†} |
| All patients | 49 (100) | 29/49 (59) | — |
| Age | | | 1.0000 |
| <65 y | 42 (86) | 25/42 (60) | |
| ≥65 y | 7 (14) | 4/7 (57) | |
| Gender, n (%) | | | 0.5579 |
| Female | 28 (57) | 18/28 (64) | |
| Male | 21 (43) | 11/21 (52) | |
| Baseline platelet count, n (%) | | | 0,7707 |
| ≤15 × 10⁹/L | 27 (55) | 15/27 (56) | |
| >15 × 10⁹/L | 22 (45) | 14/22 (64) | |
| Duration of ITP | | | 0.2008 |
| <3 y | 14 (29) | 6/14 (43) | |
| ≥3 y | 35 (71) | 23/35 (66) | |
| Prior number of ITP treatments | | | 0.1054 |
| <3 | 7 (14) | 2/7 (29) | |
| ≥3 | 42 (86) | 27/42 (64) | |
| Prior ITP medication | | — | — |
| Corticosteroids | 47 (96) | | |
| TPO-RA | 38 (78) | | |
| Prior splenectomy | | | 0.75.36 |
| No | 35 (71) | 20/35 (57) | |
| Yes | 14 (29) | 9/14 (64) | |
| Prior antidepressant medication | | | 0.5593 |
| No | 46 (94) | 28/46 (63) | |
| Yes | 3 (6) | 1/3 (33) | |
| Presence of hypertension | | | 1.0000 |
| No | 32 (65) | 19/32 (59) | |
| Yes | 17 (35) | 10/17 (59) | |
| Presence of depression/anxiety | | | 1.0000 |
| No | 41 (84) | 24/41 (59) | |
| Yes | 8 (16) | 5/8 (63) | |
| Presence of diabetes | | | 1.0000 |
| No | 42 (86) | 25/42 (60) | |
| Yes | 7 (14) | 4/7 (57) | |
| Concomitant CS^{‡} | | | 0.2402 |
| No | 28 (57) | 19/28 (68) | |
| Yes | 21 (43) | 10/21 (48) | |
| Concomitant TPO-RA^{‡} | | | 0.0450 |
| No | 28 (57) | 13/28 (46) | |
| Yes | 21 (43) | 16/21 (76) | |

*Defined as any cognitive impairment referring to any 2 or more of DET, IDN, OCL, and ONB having a score ≤−1. Percentages were calculated as number of patients with cognitive impairment divided by the number of patients within that group at baseline.
^{†}P-values from Fisher exact test comparing between impaired patient groups.
^{‡}Patients were receiving concomitant ITP medication at baseline of the study and prior to treatment initiation

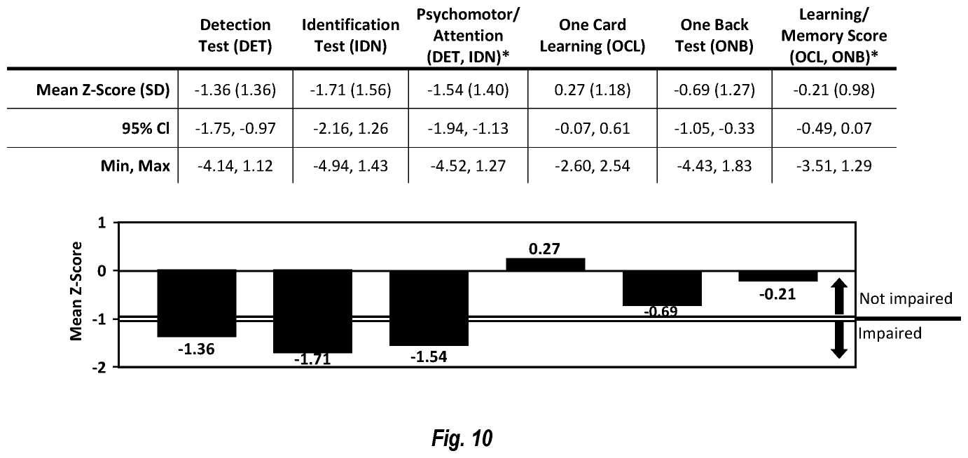

The group mean Z-scores (±95% CI) for the individual CBB tests, and for the composite scores, are shown in FIG. 10 and Table 9. As the group means are presented as performance scores standardized relative to healthy normative data (the mean of which is indicated by the solid horizontal line at mean z=0), the estimated mean provides an index of the magnitude of impairment for each aspect of the cognitive tests. The estimated mean z-scores can be inferred to be statistically significantly different from healthy controls when the associated 95% CI does not include zero (American Psychiatric Association D-TF. Diagnostic and Statistical Manual of Mental Disorders: DSM-5 (5^{th} ed); Maruff et al. 2013).

Group mean Z-scores on the detection and identification tests were both negative, large in magnitude, statistically different from healthy control data (i.e., the 95% CI did not include zero), and could be classified as clinically important. Accordingly, the mean Z-score for the psychomotor function/attention composite, which is based on these two test scores, was also negative, large in magnitude, statistically different from age-matched normative data, and clinically meaningful.

For the one back test, performance was also negative and statistically different from age-matched normative data; however, the magnitude of the impairment was moderate and thus could not be classified as clinically important. For the one card learning test, performance was slightly positive but not statistically different from age-matched normative data. Accordingly, the learning/working memory composite score, based on the scores from the one card learning and one back tests, was also small, not statistically different from age-matched normative data, and insufficient for classification as clinically important.

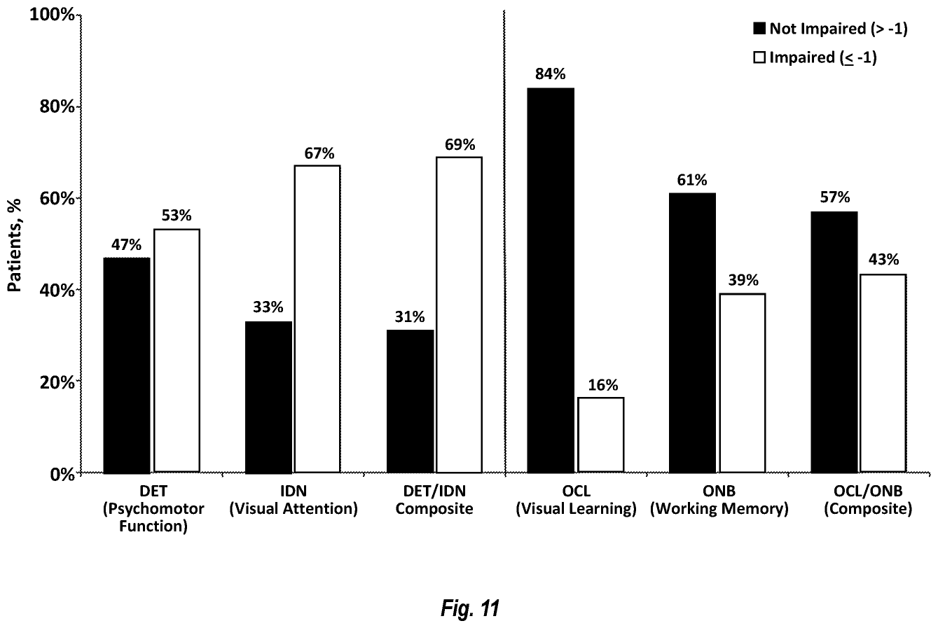

To provide a better understanding of the extent to which individual patients with ITP exhibit impairment in tests of specific aspects of cognition, the performance score for each CBB test was classified as being abnormal if the score was ≤−1 (indicating cognitive impairment). The percentage of patients with abnormal scores on each test is shown in FIG. 11. In general, the level of individual impairment followed the same pattern as the group mean scores, with top rates for attention (67% of patients), followed by psychomotor function (53% of patients), working memory (39% of patients), and visual learning (16% of patients) tests. The two composite scores were used to classify clinically important cognitive impairment in individual patients, where a score of ≤−1 on the psychomotor function/attention composite or the learning/working memory composite was classified as clinically important cognitive impairment. Using this criterion, composite psychomotor function/attention impairment was greater (69% of patients) than learning/memory (43% of patients). Additionally, the percentage of patients with psychomotor function/attention impairment was consistently higher than the percentage of patients with learning/memory impairment. Overall, 43% of patients were defined as impaired with a Z-score of ≤−1 on either of the individual tests for psychomotor function/attention and for learning/working memory. Six (12%) patients with ITP showed impairment on both of the one card learning and one back tests.

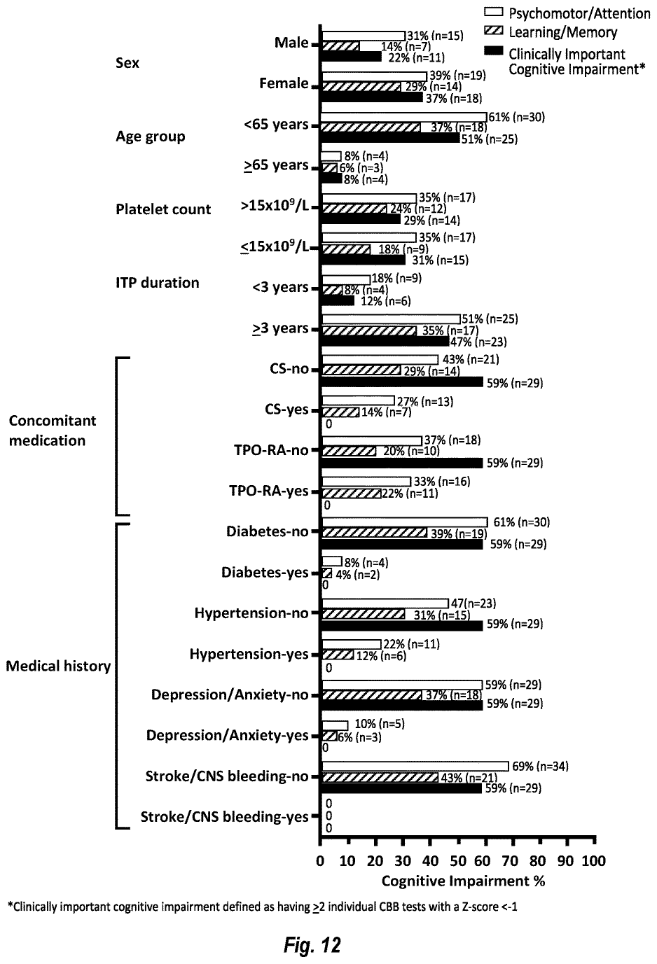

More than half of patients aged <65 years, with no concomitant ITP medication, and those with no medical history had “clinically important cognitive impairment,” which was defined as having ≥2 individual CBB tests with a Z-score<−1 (FIG. 12).

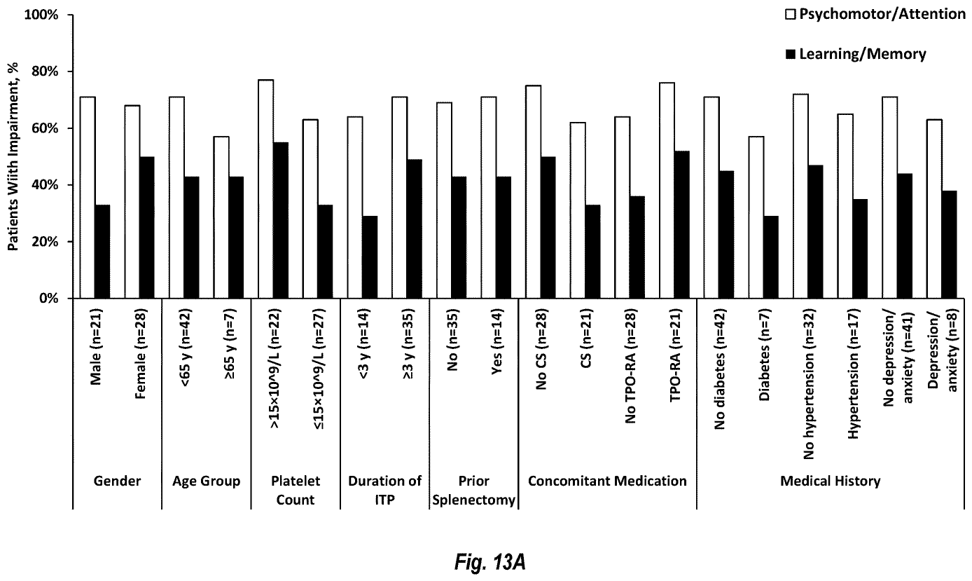

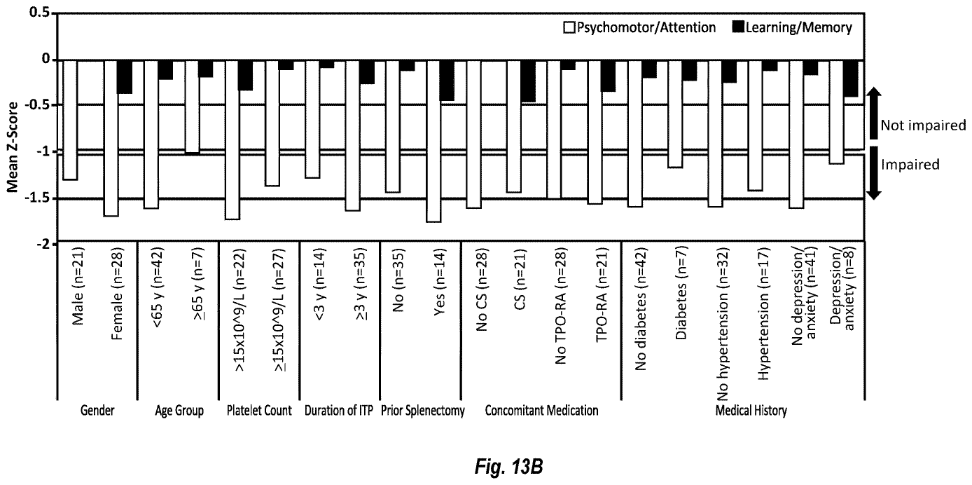

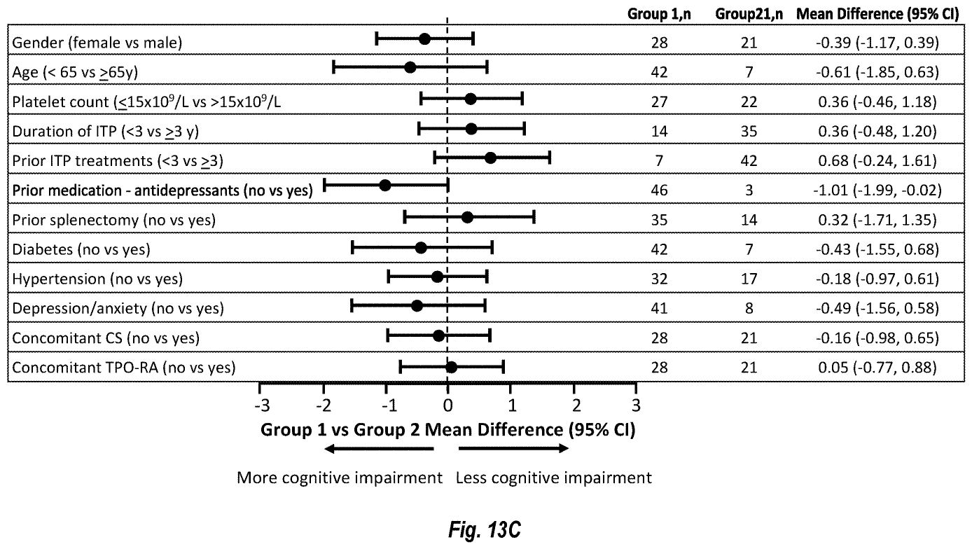

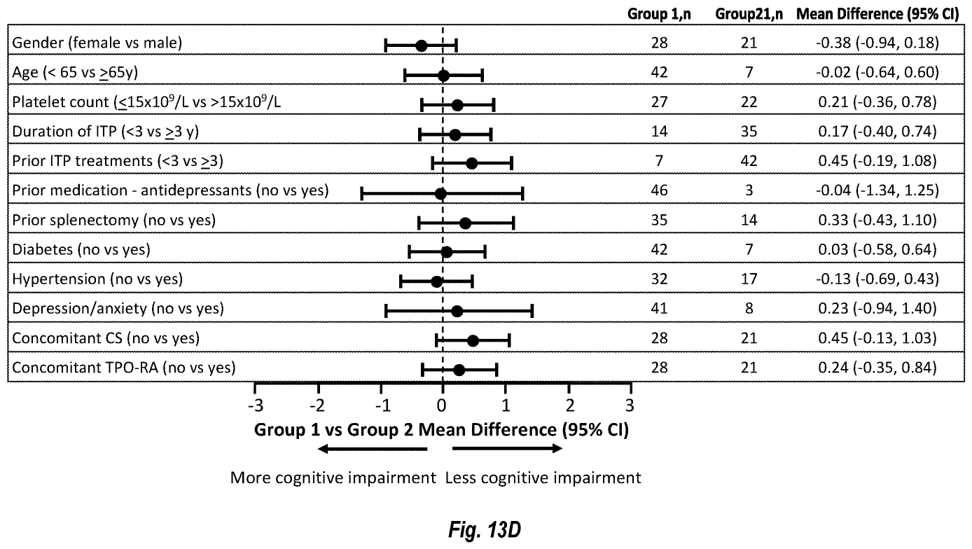

The study next assessed the relationship between ITP patient demographics or disease-related factors and performance on composite scores for psychomotor function/attention and learning/working memory (see FIGS. 12 and 13A-D). The extent to which a group mean was different between individuals with and without the baseline factor was determined on the basis of whether the associated 95% CI contained zero. For performance on the psychomotor function/attention composite, the only comparison for which the 95% CI did not include zero was for history of antidepressant use, where there was a small number of patients in the group who had received prior antidepressants (n=3; FIG. 13C). The psychomotor function/attention composite mean difference between groups favored less cognitive impairment (i.e., mean above 0) for patients with ITP who had higher platelet counts (>15×10⁹/L), longer duration of ITP (≥3 y), a higher number of prior ITP treatments (≥3), and prior splenectomy, although in each case these benefits were not significant (i.e., 95% CI included zero). For the learning/working memory composite score (see FIG. 13B), the mean difference between groups for all comparisons was similar between groups, with a slight trend toward less cognitive impairment (i.e., above 0), similar to the psychomotor function/attention composite findings, with both favoring higher platelet counts (>15×10⁹/L), longer duration of ITP (≥3 y), a higher number of prior ITP treatments (≥3), and prior splenectomy, along with patients who were receiving concomitant CS or TPO-RA.

Linear regression analyses showed that on average, the psychomotor/attention score increased with age (slope 0.02), suggesting less impairment, whereas learning/memory worsened slightly (slope −0.005) with age. Both composite scores increased with each year of ITP duration (slope 0.01 for both), indicating less impairment, but decreased with baseline platelet count increase by 1×10⁹/L (psychomotor function/attention slope −0.03, learning/memory slope −0.009), indicating more impairment.

TABLE 9
Baseline Cognition Data in Patients with Baseline CBB Assessments (N = 49).

| | Detection | Identifi- | Psychomot | One Card | One Back | |
|---|---|---|---|---|---|---|
| | Test | cation Test | or/ | Learning | Test | Learning/ |
| | (DET) | (IDN) | Attention* | (OCL) | (ONB) | Memory* |
| Mean Z — | 1.36 (1.36) | −1.71 (1.56) | −1,54 (1.40) | 0.27 (1.18) | −0.69 (1.27) | −0.21 (0.98) |
| score (SD) | | | | | | |
| 95% CI | −1.75, −0.97 | −2.16, −1.26 | −1.94, −1.13 | −0.07, 0.61 | −1.05, −0.33 | −0.49, 0.07 |
| Min, Max | −4.14, 1,12 | −4.94, 1,43 | −4,52, 1,27 | −2.60, 2 54 | −4.43, 1.83 | −3.51, 1.29 |
| Impairment, | 26 (53) | 33 (67) | 34 (69) | 8 (16) | 19 (39) | 21 (43) |
| n (%)† | | | | | | |
| Distribution, | | | | | | |
| n (%) | | | | | | |
| Not | 23 (47) | 16 (33) | — | 41 (84) | 30 (61) | — |
| impaired | | | | | | |
| ≥−1.0 | | | | | | |
| Impaired | 26 (53) | 33 (67) | — | 8 ( 16) | 19 (39) | — |
| <−1 to ≥−2 | 11 (22) | 14 (29) | — | 6 (12) | 14 (29) | — |
| <−2 to ≥−3 | 7 (14) | 9(18) | — | 2 (4) | 3 (6) | — |
| <−3 | 8 (16) | 10 (20) | — | 0 (0) | 2 (4) | — |

*For summaries of composite scores, the mean (DET, IDN) was used for
Psychomotor/Attention and mean (OCL, ONB) for Learning/Memory, respectively
†Cognitive impairment implies a Z-score <−1.

This report provides evidence of cognitive impairment in patients with ITP. Indeed, clinically important cognitive dysfunction occurred in nearly 50% of ITP patients, with the majority of impairment manifesting in attentional functions. Cognitive impairment was moderate in magnitude for psychomotor function/attention, and mild for learning/memory. The observed impairment had no significant correlation with biological/clinical characteristics and was unrelated to concomitant use of corticosteroids or TPO-RA.

From the perspective of magnitude alone, the attentional function impairment observed in ITP patients was equivalent to or greater in magnitude than the impairment observed in age-matched patients with mild traumatic brain injury, moderate-to-severe depressive disorder, or chronic fatigue syndrome (Davis M T et al., 2017; Joustra M L et al., 2022; Maruff P et al., 2009). Moreover, the nature and magnitude of impairment observed in the ITP group is consistent with a vascular cognitive disorder (Sachdev P et al., 2014).

### Embodiments

Non-limiting embodiments of the disclosure include:
1. A method of treating immune thrombocytopenia (ITP) in a patient in need thereof comprising administering to the patient in need thereof a therapeutically effective amount of rilzabrutinib, wherein the patient in need thereof has at least one characteristic chosen from:
   a. a Z-score of ≤−1 on the Cogstate Brief Battery (CBB) detection test (DET) relative to age-matched normative data;
   b. a Z-score of ≤−1 on the CBB identification test (IDN) relative to age-matched normative data;
   c. a Z-score of ≤−1 for composite DET and IDN scores relative to age-matched normative data;
   d. a Z-score of ≤−1 on the CBB one card learning test (OCL) relative to age-matched normative data;
   e. a Z-score of ≤−1 on the CBB one back test (ONB) relative to age-matched normative data; and
   f. a Z-score of ≤−1 for composite OCL and ONB scores relative to age-matched normative data.
2. A method of treating immune thrombocytopenia (ITP) in a patient in need thereof comprising administering to the patient in need thereof a therapeutically effective amount of rilzabrutinib, wherein the patient in need thereof has at least two characteristics chosen from:
   a. a Z-score of ≤−1 on the Cogstate Brief Battery (CBB) detection test (DET) relative to age-matched normative data;
   b. a Z-score of ≤−1 on the CBB identification test (IDN) relative to age-matched normative data;
   c. a Z-score of ≤−1 for composite DET and IDN scores relative to age-matched normative data;
   d. a Z-score of ≤−1 on the CBB one card learning test (OCL) relative to age-matched normative data;
   e. a Z-score of ≤−1 on the CBB one back test (ONB) relative to age-matched normative data; and
   f. a Z-score of ≤−1 for composite OCL and ONB scores relative to age-matched normative data.
3. The method according to any one of Embodiments 1 or 2, wherein the patient in need thereof has a Z-score of <−2 on the DET relative to age-matched normative data.
4. The method according to any one of Embodiments 1-3, wherein the patient in need thereof has a Z-score of <−3 on the DET relative to age-matched normative data.
5. The method according to any one of Embodiments 1-4, wherein the patient in need thereof has a Z-score of <−2 on the IDN relative to age-matched normative data.
6. The method according to any one of Embodiments 1-5, wherein the patient in need thereof has a Z-score of <−3 on the IDN relative to age-matched normative data.
7. The method according to any one of Embodiments 1-6, wherein the patient in need thereof has a Z-score of <−2 for composite DET and IDN scores relative to age-matched normative data.
8. The method according to any one of Embodiments 1-7, wherein the patient in need thereof has a Z-score of <−3 for composite DET and IDN scores relative to age-matched normative data.
9. The method according to any one of Embodiments 1-8, wherein the patient in need thereof has a Z-score of <−2 on the OCL relative to age-matched normative data.
10. The method according to any one of Embodiments 1-9, wherein the patient in need thereof has a Z-score of <−3 on the OCL relative to age-matched normative data.
11. The method according to any one of Embodiments 1-10, wherein the patient in need thereof has a Z-score of <−2 on the ONB relative to age-matched normative data.
12. The method according to any one of Embodiments 1-11, wherein the patient in need thereof has a Z-score of <−3 on the ONB relative to age-matched normative data.
13. The method according to any one of Embodiments 1-12, wherein the patient in need thereof has a Z-score of <−2 for composite OCL and ONB scores relative to age-matched normative data.
14. The method according to any one of Embodiments 1-13, wherein the patient in need thereof has a Z-score of <−3 for composite OCL and ONB scores relative to age-matched normative data.
15. The method according to any one of Embodiments 1-14, wherein the patient in need thereof has a platelet count of count<33,000/μL on two occasions no less than 7 days apart in the 15 days prior to beginning study treatment.
16. The method according to any one of Embodiments 1-15, wherein the patient in need thereof has a history of response (two or more platelet counts≥50,000/μL with an increase of ≥20,000/μL) to at least one prior line of therapy.
17. The method according to Embodiment 16, wherein the at least one prior line of therapy is chosen from a splenectomy, intravenous immunoglobin (IVIG), corticosteroids, anti-D immunoglobulin therapy, and immunosuppressive drugs.
18. The method according to any one of Embodiments 1-17, wherein the patient in need thereof achieves at least one platelet count of at least 20,000/μL above a baseline platelet count, wherein the baseline platelet count is the mean of two platelet counts taken on two occasions no less than 7 days apart in the 15 days prior to beginning treatment and a third count taken on the first day of the study.
19. The method according to any one of Embodiments 1-18, wherein the patient in need thereof achieves at least two platelet counts of at least 20,000/μL above the baseline platelet count.
20. The method according to any one of Embodiments 1-19, wherein the patient in need thereof achieves at least two consecutive platelet counts of at least 20,000/μL above the baseline platelet count.
21. The method according to any one of Embodiments 19 or 20, wherein at least two consecutive platelet counts of at least 20,000/μL above the baseline platelet count are separated by at least five days, for example at least seven days.
22. The method according to any one of Embodiments 1-21, wherein the patient in need thereof achieves a platelet count of at least 50,000/μL.
23. The method according to any one of Embodiments 1-22, wherein the patient in need thereof achieves at least two platelet counts of at least 50,000/μL.
24. The method according to Embodiment 23, wherein the patient in need thereof achieves at least two consecutive platelet counts of at least 50,000/μL.
25. The method according to any one of Embodiments 23 or 24, wherein the at least two consecutive platelet counts of at least 50,000/μL are separated by at least five days, for example at least seven days.
26. The method according to any one of Embodiments 1-25, wherein the patient in need thereof achieves at least one platelet count of at least 50,000/μL within eight days of initiating treatment.
27. The method according to any one of Embodiments 22-26, wherein the patient in need thereof does not receive rescue medication during the four weeks prior to the most recent platelet count of at least 50,000/μL.
28. The method according to any one of Embodiments 1-27, wherein the patient in need thereof has had ITP for three or more years.
29. The method according to any one of Embodiments 1-28, wherein the patient in need thereof has a history of taking at least one prior ITP therapy prior to the start of the treatment period.
30. The method according to any one of Embodiments 1-29, wherein the patient in need thereof has platelet counts between 2,000/μL and 33,000/μL on two occasions no less than 7 days apart and within 15 days prior to the start of the treatment period.
31. The method according to any one of Embodiments 1-30, wherein the patient in need thereof has platelet counts≤15,000/u L on two occasions no less than 7 days apart and within 15 days prior to the start of the treatment period.
32. The method according to any one of Embodiments 1-30, wherein the patient in need thereof has platelet counts>15,000/μL on two occasions no less than 7 days apart and within 15 days prior to the start of the treatment period.
33. The method according to any one of Embodiments 1-32, wherein the patient in need thereof has a history of response to at least one prior line of therapy, wherein at least one prior therapy is chosen from a splenectomy, intravenous immunoglobin (IVIG), corticosteroids, anti-D immunoglobulin therapy, immunosuppressive drugs, TPO-RAs, and rituximab.
34. The method according to any one of Embodiments 1-33, wherein the patient in need thereof had a splenectomy prior to the start of the treatment period.
35. The method according to any one of Embodiments 1-33, wherein the patient in need thereof has a history of taking intravenous immunoglobin (IVIG) prior to the start of the treatment period.
36. The method according to any one of Embodiments 1-33, wherein the patient in need thereof has a history of taking corticosteroids prior to the start of the treatment period.
37. The method according to any one of Embodiments 1-33, wherein the patient in need thereof has a history of taking anti-D immunoglobulin therapy prior to the start of the treatment period.
38. The method according to any one of Embodiments 1-33, wherein the patient in need thereof has a history of taking at least one immunosuppressive drug prior to the start of the treatment period.
39. The method according to Embodiment 38, wherein the at least one immunosuppressive drug is selected from fostamatinib, mycophenolate mofetil (MMF), and cyclosporine.
40. The method according to any one of Embodiments 1-33, wherein the patient in need thereof has a history of taking at least one TPO-RA prior to the start of the treatment period.
41. The method according to Embodiment 40, wherein the at least one TPO-RA is selected from rTPO, romiplostim, eltrombopag, and avatrombopag.
42. The method according to any one of Embodiments 1-33, wherein the patient in need thereof has a history of taking rituximab prior to the start of the treatment period.
43. The method according to any one of Embodiments 33-42, wherein the response to the prior ITP therapy comprised a platelet count of ≥50,000/μL.
44. The method of any one of Embodiments 1-43, wherein the patient in need thereof receives treatment with at least one TPO-RA.
45. The method of Embodiment 44, wherein the at least one TPO-RA is selected from rTPO, romiplostim, eltrombopag, and avatrombopag.
46. The method according to any one of Embodiments 1-45, wherein the patient in need thereof has primary ITP.
47. The method according to any one of Embodiments 1-45, wherein the patient in need thereof has secondary ITP.
48. The method according to any one of Embodiments 1-45, wherein the patient in need thereof does not have chronic ITP.
49. The method according to any one of Embodiments 1-45, wherein the patient in need thereof has persistent ITP.
50. The method according to any one of Embodiments 1-45, wherein the patient in need thereof has chronic ITP.
51. The method according to any one of Embodiments 1-45, wherein the patient in need thereof has relapsing ITP.
52. The method according to any one of Embodiments 1-45, wherein the patient in need thereof has refractory ITP.
53. The method according to any one of Embodiments 1-52, wherein the treatment period is at least 8 days.
54. The method according to any one of Embodiments 1-52, wherein the treatment period is at least 28 days.
55. The method according to any one of Embodiments 1-52, wherein the treatment period is at least 84 days.
56. The method according to any one of Embodiments 1-52, wherein the treatment period is at least 169 days.
57. The method according to any one of Embodiments 1-56, comprising administering to the patient in need thereof 400 mg of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day.
58. The method according to any one of Embodiments 1-57, wherein the at least one compound consists of at least one compound chosen from the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof.
59. The method according to any one of Embodiments 1-57, wherein the at least one compound consists of at least one compound chosen from the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof.
60. The method according to any one of Embodiments 1-57, wherein the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt of the foregoing.
61. The method according to any one of Embodiments 1-56, comprising administering to the patient in need thereof 400 mg of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile twice a day.
62. The method according to any one of Embodiments 1-56 or 61, wherein the at least one compound is the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.
63. The method according to any one of Embodiments 1-56 or 61, wherein the at least one compound is the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.
64. The method according to any one of Embodiments 1-56 or 61, wherein the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.
65. The method according to any one of Embodiments 1-64, wherein the at least one compound is orally administered to the patient.
66. The method according to any one of Embodiments 1-65, wherein the at least one compound is administered to the patient in the form of at least one tablet.
67. The method according to any one of Embodiments 1-66, wherein the at least one compound is administered with water.
68. The method according to any one of Embodiments 1 or 2 or 5-67, wherein the patient in need thereof has a Z-score of <−1 on the DET relative to age-matched normative data.
69. The method according to any one of Embodiments 1~4 or 7-68, wherein the patient in need thereof has a Z-score of <−1 on the IDN relative to age-matched normative data.
70. The method according to any one of Embodiments 1-6 or 9-69, wherein the patient in need thereof has a Z-score of <−1 for composite DET and IDN scores relative to age-matched normative data.
71. The method according to any one of Embodiments 1-8 or 11-70, wherein the patient in need thereof has a Z-score of <−1 on the OCL relative to age-matched normative data.
72. The method according to any one of Embodiments 1-10 or 13-71, wherein the patient in need thereof has a Z-score of <−1 on the ONB relative to age-matched normative data.
73. The method according to any one of Embodiments 1-12 or 15-72, wherein the patient in need thereof has a Z-score of <−1 for composite OCL and ONB scores relative to age-matched normative data.

Claims or descriptions that include “or” or “and/or” between at least one members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all the group members are present in, employed in, or otherwise relevant to a given product or process.

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

1. A method of treating immune thrombocytopenia (ITP) in a patient in need thereof comprising administering to the patient in need thereof a therapeutically effective amount of rilzabrutinib, wherein the patient in need thereof has at least one characteristic chosen from:
a. a Z-score of ≤−1 on the Cogstate Brief Battery (CBB) detection test (DET) relative to age-matched normative data;
b. a Z-score of ≤−1 on the CBB identification test (IDN) relative to age-matched normative data;
c. a Z-score of ≤−1 for composite DET and IDN scores relative to age-matched normative data;
d. a Z-score of ≤−1 on the CBB one card learning test (OCL) relative to age-matched normative data;
e. a Z-score of ≤−1 on the CBB one back test (ONB) relative to age-matched normative data; and
f. a Z-score of ≤−1 for composite OCL and ONB scores relative to age-matched normative data.

2. A method of treating immune thrombocytopenia (ITP) in a patient in need thereof comprising administering to the patient in need thereof a therapeutically effective amount of rilzabrutinib, wherein the patient in need thereof has at least two characteristics chosen from:
a. a Z-score of ≤−1 on the Cogstate Brief Battery (CBB) detection test (DET) relative to age-matched normative data;
b. a Z-score of ≤−1 on the CBB identification test (IDN) relative to age-matched normative data;
c. a Z-score of ≤−1 for composite DET and IDN scores relative to age-matched normative data;
d. a Z-score of ≤−1 on the CBB one card learning test (OCL) relative to age-matched normative data;
e. a Z-score of ≤−1 on the CBB one back test (ONB) relative to age-matched normative data; and
f. a Z-score of ≤−1 for composite OCL and ONB scores relative to age-matched normative data.

3. The method of claim 1, wherein the patient in need thereof has a Z-score of <−2 on the DET relative to age-matched normative data.

4. The method of claim 1, wherein the patient in need thereof has at least one characteristic chosen from:
(a) a Z-score of <−3 on the DET relative to age-matched normative data;
(b) a Z-score of <−3 on the IDN relative to age-matched normative data;
(c) a Z-score of <−3 for composite DET and IDN scores relative to age-matched normative data;
(d) a Z-score of <−3 on the OCL relative to age-matched normative data;
(e) a Z-score of <−3 on the ONB relative to age-matched normative data; and
(f) a Z-score of <−3 for composite OCL and ONB scores relative to age-matched normative data.

5. The method of claim 1, wherein the patient in need thereof has a Z-score of <−2 on the IDN relative to age-matched normative data.

6. (canceled)

7. The method of claim 1, wherein the patient in need thereof has a Z-score of <−2 for composite DET and IDN scores relative to age-matched normative data.

8. (canceled)

9. The method of claim 1, wherein the patient in need thereof has a Z-score of <−2 on the OCL relative to age-matched normative data.

10. (canceled)

11. The method of claim 1, wherein the patient in need thereof has a Z-score of <−2 on the ONB relative to age-matched normative data.

12. (canceled)

13. The method of claim 1, wherein the patient in need thereof has a Z-score of <−2 for composite OCL and ONB scores relative to age-matched normative data.

14. (canceled)

15. The method of claim 1, wherein at least one of the following occurs:
(a) the patient in need thereof has a platelet count of count<33,000/μL on two occasions no less than 7 days apart in the 15 days prior to beginning study treatment;
(b) the patient in need thereof has a history of response (two or more platelet counts≥50,000/μL with an increase of ≥20,000/μL) to at least one prior line of therapy;
(c) the patient in need thereof has a history of response (two or more platelet counts≥50,000/μL with an increase of ≥20,000/μL) to at least one prior line of therapy chosen from a splenectomy, intravenous immunoglobin (IVIG), corticosteroids, anti-D immunoglobulin therapy, and immunosuppressive drugs;
(d) the patient in need thereof has platelet counts between 2,000/μL and 33,000/μL on two occasions no less than 7 days apart and within 15 days prior to the start of the treatment period;
(e) the patient in need thereof has platelet counts≤15,000/μL on two occasions no less than 7 days apart and within 15 days prior to the start of the treatment period; and
(f) the patient in need thereof has platelet counts≥15,000/μL on two occasions no less than 7 days apart and within 15 days prior to the start of the treatment period.

16.-17. (canceled)

18. The method of claim 1, wherein a baseline platelet count is the mean of two platelet counts taken on two occasions no less than 7 days apart in the 15 days prior to beginning treatment and a third count taken on the first day of the study, and wherein at least one of the following occurs:
(a) the patient in need thereof achieves at least one platelet count of at least 20,000/μL above the baseline platelet count;
(b) the patient in need thereof achieves at least two platelet counts of at least 20,000/μL above the baseline platelet count;
(c) the patient in need thereof achieves at least two consecutive platelet counts of at least 20,000/μL above the baseline platelet count;
(d) the patient in need thereof achieves at least two platelet counts of at least 20,000/μL above the baseline platelet count separated by at least five days or at least seven days; and
(e) the patient in need thereof achieves at least two consecutive platelet counts of at least 20,000/μL above the baseline platelet count separated by at least five days or at least seven days.

19.-21. (canceled)

22. The method of claim 1, wherein at least one of the following occurs:
(a) the patient in need thereof achieves a platelet count of at least 50,000/μL;
(b) the patient in need thereof achieves at least two platelet counts of at least 50,000/μL;
(c) the patient in need thereof achieves at least two consecutive platelet counts of at least 50,000/μL;
(d) the patient in need thereof achieves at least two platelet counts of at least 50,000/μL separated by at least five days or at least seven days;
(e) the patient in need thereof achieves at least two consecutive platelet counts of at least 50,000/μL separated by at least five days or at least seven days;
(f) the patient in need thereof achieves at least one platelet count of at least 50,000/μL within eight days of initiating treatment;
(g) the patient in need thereof achieves a platelet count of at least 50,000/μL and does not receive rescue medication during the four weeks prior to the most recent platelet count of at least 50,000/μL;
(h) the patient in need thereof achieves at least two platelet counts of at least 50,000/μL and does not receive rescue medication during the four weeks prior to the most recent platelet count of at least 50,000/μL;
(i) the patient in need thereof achieves at least two consecutive platelet counts of at least 50,000/μL and does not receive rescue medication during the four weeks prior to the most recent platelet count of at least 50,000/μL;
(j) the patient in need thereof achieves at least two platelet counts of at least 50,000/μL separated by at least five days or at least seven days and does not receive rescue medication during the four weeks prior to the most recent platelet count of at least 50,000/μL;
(k) the patient in need thereof achieves at least two consecutive platelet counts of at least 50,000/μL separated by at least five days or at least seven days and does not receive rescue medication during the four weeks prior to the most recent platelet count of at least 50,000/μL; and
(l) the patient in need thereof achieves at least one platelet count of at least 50,000/μL within eight days of initiating treatment and does not receive rescue medication during the four weeks prior to the most recent platelet count of at least 50,000/μL.

23.-32. (canceled)

33. The method of claim 1, wherein the patient in need thereof has a history of response to at least one prior line of therapy, and wherein at least one of the following occurs:
(a) the at least one prior therapy is chosen from a splenectomy, intravenous immunoglobin (IVIG), corticosteroids, anti-D immunoglobulin therapy, immunosuppressive drugs, TPO-RAs, and rituximab; and
(b) the response to the prior ITP therapy comprised a platelet count of ≥50,000/μL.

34. The method of claim 1, wherein at least one of the following occurs:
(a) the patient in need thereof had a splenectomy prior to the start of the treatment period;
(b) the patient in need thereof has a history of taking intravenous immunoglobin (IVIG), corticosteroids, anti-D immunoglobulin, at least one immunosuppressive drug, at least one TPO-RA, or rituximab prior to the start of the treatment period;
(c) the patient in need thereof has a history of taking at least one immunosuppressive drug selected from fostamatinib, mycophenolate mofetil (MMF), and
cyclosporine;
(d) the patient in need thereof has a history of taking at least one TPO-RA selected from rTPO, romiplostim, eltrombopag, and avatrombopag;
(e) the patient in need thereof has a history of taking at least one prior ITP therapy prior to the start of the treatment period; and
(f) the patient in need thereof has had ITP for three or more years.

35.-43. (canceled)

44. The method of claim 1, wherein;
(a) the patient in need thereof receives treatment with at least one TPO-RA; or
(b) the patient in need thereof receives treatment with at least one TPO-RA selected from rTPO, romiplostim, eltrombopag, and avatrombopag.

45. (canceled)

46. The method of claim 1, wherein at least one of the following occurs:
(a) the patient in need thereof has primary ITP;
(b) the patient in need thereof has secondary ITP;
(c) the patient in need thereof does not have chronic ITP;
(d) the patient in need thereof has persistent ITP;
(e) the patient in need thereof has chronic ITP;
(f) the patient in need thereof has relapsing ITP; and
(g) the patient in need thereof has refractory ITP.

47.-52. (canceled)

53. The method of claim 1, wherein the treatment period is at least 8 days, at least 28 days, at least 84 days, or at least 169 days.

54.-56. (canceled)

57. The method of claim 1, comprising:
(a) administering to the patient in need thereof 400 mg of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day; or
(b) administering to the patient in need thereof 400 mg of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day, wherein:
(i) the at least one compound consists of at least one compound chosen from the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof;
(ii) the at least one compound consists of at least one compound chosen from the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof; or
(iii) the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt of the foregoing.

58.-60. (canceled)

61. The method of claim 1, comprising:
(a) administering to the patient in need thereof 400 mg of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile twice a day;
(b) administering to the patient in need thereof at least one compound comprising 400 mg of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile twice a day, wherein:
(i) the at least one compound is the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile;
(ii) the at least one compound is the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile; or
(iii) the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.

62.-64. (canceled)

65. The method of claim 1, wherein at least one of the following occurs:
(a) the rilzabrutinib is orally administered to the patient;
(b) the rilzabrutinib is administered to the patient in the form of at least one tablet; and
(c) the rilzabrutinib is administered with water.

66.-67. (canceled)
